(19) United States
(12) Patent Application Publication
(10) Pub. No.: US 2006/0188976 A1
(43) Pub. Date: Aug. 24, 2006

(54) Mutant protein having the peptide-synthesizing activity

(75) Inventors: Rie Takeshita, Kawasaki-shi (JP); Isao Abe, Kawasaki-shi (JP); Masakazu Sugiyama, Kawasaki-shi (JP); Kenzo Yokozeki, Kawasaki-shi (JP); Seiichi Hara, Kawasaki-shi (JP); Sonoko Suzuki, Kawasaki-shi (JP); Shunichi Suzuki, Kawasaki-shi (JP); Kunihiko Watanabe, Kawasaki-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: AJINOMOTO CO. INC, Tokyo (JP)

(21) Appl. No.: 11/311,467

(22) Filed: Dec. 20, 2005

Related U.S. Application Data
(60) Provisional application No. 60/638,370, filed on Dec. 27, 2004.

(30) Foreign Application Priority Data
Dec. 20, 2004 (JP) 2004-368503

(51) Int. Cl. C12N 9/48; C07H 21/04; C12P 21/06; C12N 1/21; C12N 15/74

(52) U.S. Cl. 435/212; 435/69.1; 435/252.3; 435/471; 536/23.2

(57) ABSTRACT

The present invention is to provide an excellent peptide-synthesizing protein and a method for efficiently producing a peptide. A peptide is synthesized by reacting an amine component and a carboxy component in the presence of at least one of proteins shown in the following (I) and (II): (I) The mutant protein having the amino acid sequence containing one or more mutations of the above mutations 1 to 38 in the amino acid sequence described in SEQ ID NO:2; and (II) The mutant protein having the amino acid sequence containing one or more mutations selected from the group consisting of substitution, deletion, insertion, addition and inversion at positions other than one or more mutation positions of the above mutation 1 to 38 in the mutant protein described in the above (I), and having a peptide-synthesizing activity.

### FIELD OF INVENTION

The present invention relates to a mutant protein having a peptide-synthesizing activity, and more particularly relates to a mutant protein having an excellent peptide-synthesizing activity and a method for producing a peptide using this protein.

### PRIOR ART

Peptides have been used in a variety of fields such as pharmaceuticals and foods. For example, L-alanyl-L-glutamine is widely used as a component for infusions and serum-free media taking advantage of its higher stability and water-solubility than that of L-glutamine.

Peptides have hitherto been produced by chemical synthesis methods. However, the chemical synthesis has not always been satisfactory in terms of simplicity and efficiency.

On the other hand, methods for producing the peptide using an enzyme have been developed (e.g., Patent documents 1 and 2). However, the conventional enzymological method for producing the peptide still had room for improvement such as slow synthesis rate and low yield of the peptide products. In such a context, it has been desired to develop a method for efficiently producing peptides on an industrial scale.

The present inventors have already been found an enzyme derived from Sphingobacterium as an enzyme having an excellent peptide-synthesizing activity (Patent document 3).

(List of Prior Art References)

Patent document 1: EP 0278787 A1
Patent document 2: EP 359399 A1
Patent document 3: WO2004/011653

### DISCLOSURE OF THE INVENTION

It is an object of the present invention to provide a more excellent peptide-synthesizing protein and a method for efficiently producing the peptide.

As a result of an extensive study, the present inventors have successfully isolated a gene encoding a protein having a peptide-synthesizing activity derived from a microorganism belonging to genus Sphingobacterium, and have found out that modification of a specific position(s) in the amino sequence or the nucleotide sequence thereof results in production of a protein having more excellent peptide-synthesizing activity, to thereby complete the present invention. That is, the present invention provides the following proteins and methods for producing peptides using these proteins.

[1] A mutant protein having an amino acid sequence comprising one or two or more mutations selected from any of the following mutations 1 to 38: mutation 1: F207V, mutation 2: Q441E, mutation 3: K83A, mutation 4: A301V, mutation 5: V257I, mutation 6: A537G, mutation 7: A324V, mutation 8: N607K, mutation 9: D313E, mutation 10: Q229H, mutation 11: M208A, mutation 12: E551K, mutation 13: F207H, mutation 14: T72A, mutation 15: A137S, mutation 16: L439V, mutation 17: G226S, mutation 18: D619E, mutation 19: Y339H, mutation 20: W327G, mutation 21: V184A, mutation 22: V184C, mutation 23: V184G, mutation 24: V184I, mutation 25: V184L, mutation 26: V184M, mutation 27: V184P, mutation 28: V184S, mutation 29: V184T, mutation 30: Q441K, mutation 31: N442K, mutation 32: D203N, mutation 33: D203S, mutation 34: F207A, mutation 35: F207S, mutation 36: Q441N, mutation 37: F207T, and mutation 38: F207I in an amino acid sequence of SEQ ID NO:2.

[2] The mutant protein according to [1] above further having one or several amino acid mutations selected from the group consisting of substitution, deletion, insertion, addition and inversion at positions other than one or two or more mutation positions of said mutations 1 to 38, and having a peptide-synthesizing activity.

[3] The mutant protein according to [1] or [2] above comprising at least the mutation 2.

[4] The mutant protein according to any one of [1] to [3] above comprising at least the mutation 14.

[5] A polynucleotide encoding an amino acid sequence of the mutant protein according to any one of [1] to [4] above.

[6] A recombinant polynucleotide comprising the polynucleotide according to [5] above.

[7] A transformed microorganism comprising the recombinant polynucleotide according to [6] above.

[8] A method for producing a mutant protein wherein the transformed microorganism according to [7] above is cultured in a medium and said mutant protein is accumulated in the medium or the transformed microorganism.

[9] A method for producing a peptide wherein a peptide synthesizing reaction is performed in the presence of the mutant protein according to any one of [1] to [4] above.

With the present invention, the protein excellent in peptide-synthesizing activity and the method for efficiently producing the peptide are provided.

### BRIEF DESCRIPTION OF DRAWINGS

FIG. 1 is a view showing experimental results for pH stability.

FIG. 2 is a view showing experimental results for optimal reaction temperature.

FIG. 3 is a view showing experimental results for temperature stability.

### PREFERRED EMBODIMENTS OF THE INVENTION

Embodiments of the present invention will be described below along with best modes thereof.

Concerning various gene engineering techniques included below, many standard experimental manuals such as Molecular Cloning, 2nd edition, Cold Spring Harbor Press, 1989; Saibo Kogaku Handbook (Cellular Engineering Handbook) edited by Toshio Kuroki, Yodosha, 1992; and Shin Idenshi Kogaku Handbook (New Genetic Engineering Handbook), revised 3rd edition edited by Muramatsu et al., Yodosha, 1999 are available, and such techniques are feasible for those skilled in the art with reference to these literatures.

Abbreviations used herein for amino acids, peptides, nucleic acids, nucleotide sequences and the like conform to definitions by IUPAC (International Union of Pure and Applied Chemistry) or IUBMB (International Union of Biochemistry and Molecular Biology), or conventional legends used in “Guideline for the preparation of specification and others containing a base sequence and an amino acid sequence (edited by Japanese Patent Office)” and in the art. Sequence numbers used herein indicate the sequence numbers in Sequence Listing unless otherwise specified.

1. Protein Having Peptide-Synthesizing Activity of the Present Invention

A protein of the present invention is a mutant protein having an amino acid sequence into which one or more mutations of the following mutations 1 to 38 have been introduced in an amino acid sequence described in SEQ ID NO:2, and having a peptide-synthesizing activity (this protein may be referred to hereinbelow as a “mutant protein (I)”). The mutations 1 to 38 are as shown in Table 1-1.

TABLE 1-1
Table 1-1: MUTATION

| MUTATION No. | MUTATION |
|---|---|
| 1 | F207V |
| 2 | Q441E |
| 3 | K83A |
| 4 | A301V |
| 5 | V257I |
| 6 | A537G |
| 7 | A324V |
| 8 | N607K |
| 9 | D313E |
| 10 | Q229H |
| 11 | M208A |
| 12 | E551K |
| 13 | F207H |
| 14 | T72A |
| 15 | A137S |
| 16 | L439V |
| 17 | G226S |
| 18 | D619E |
| 19 | Y339H |
| 20 | W327G |
| 21 | V184A |
| 22 | V184C |
| 23 | V184G |
| 24 | V184I |
| 25 | V184L |
| 26 | V184M |
| 27 | V184P |
| 28 | V184S |
| 29 | V184T |
| 30 | Q441K |
| 31 | N442K |
| 32 | D203N |
| 33 | D203S |
| 34 | F207A |
| 35 | F207S |
| 36 | Q441N |
| 37 | F207T |
| 38 | F207I |

In the present specification, each mutation is specified by the abbreviation of an amino acid residue and a position in the amino acid sequence in SEQ ID NO:1 or 2 as shown in Table 1-1. For example, “F207V” in the mutation 1 indicates that phenylalanine at position 207 in the sequence of SEQ ID NO:2 has been substituted with valine. That is, the mutation is represented by the type of the amino acid residue in a wild type (amino acid specified by SEQ ID NO:2), the position of the amino acid residue in the amino acid sequence described in SEQ ID NO:2, and a type of the amino acid residue after being mutated. Other mutations are represented in the same way.

Each of the mutations 1 to 38 may be introduced alone or in combination of two or more. Specifically, combinations shown in Table 1-2 are preferable. The mutant protein containing at least the mutation 2: Q441E or the mutant protein containing at least the mutation 14: T72A are suitable in terms of enhancing the peptide-synthesizing activity.

TABLE 1-2
Table 1-2: MUTATION (COMBINATION
OF TWO OR MORE MUTATIONS)

| MUTATION | | ABBREVIATED |
|---|---|---|
| No. | MUTATION | NAME |
| 239 | F207V + Q441E | |
| 240 | F207V + K83A | |
| 241 | F207V + E551K | |
| 242 | K83A + Q441E | |
| 243 | M208A + E551K | |
| 244 | V257I + Q441E | |
| 245 | V257I + A537G | |
| 246 | F207V + S209A | |
| 247 | K83A + S209A | |
| 248 | K83A + F207V + Q441E | |
| 249 | L439V + F207V + Q441E | |
| 250 | A537G + F207V + Q441E | |
| 251 | A301V + F207V + Q441E | |
| 252 | G226S + F207V + Q441E | |
| 253 | V257I + F207V + Q441E | |
| 254 | D619E + F207V + Q441E | |
| 255 | Y339H + F207V + Q441E | |
| 256 | N607K + F207V + Q441E | |
| 257 | A324V + F207V + Q441E | |
| 258 | Q229H + F207V + Q441E | |
| 259 | W327G + F207V + Q441E | |
| 260 | A301V + L439V + A537G + | M7-35 |
| | N607K | |
| 261 | K83A + Q229H + A301V + | M7-46 |
| | D313E + A324V + L439V + | |
| | A537G + N607K | |
| 262 | Q229H + V257I + A301V + | M7-54 |
| | A324V + Q441E + A537G + | |
| | N607K | |
| 263 | Q229H + A301V + A324V + | M7-63 |
| | Q441E + A537G + N607K | |
| 264 | Q229H + V257I + A301V + | M7-95 |
| | D313E + A324V + Q441E + | |
| | A537G + N607K | |
| 265 | T72A + A137S + A301V + | M9-9 |
| | L439V + Q441E + A537G + | |
| | N607K | |
| 266 | T72A + A137S + A301V + | M9-10 |
| | Q441E + A537G + N607K | |
| 267 | T72A + A137S + Q229H + | M11-2 |
| | A301V + A324V + L439V + | |
| | A537G + N607K | |
| 268 | T72A + A137S + Q229H + | M11-3 |
| | A301V + A324V + L439V + | |
| | Q441E + A537G + N607K | |
| 269 | T72A + Q229H + V257I + | M12-1 |
| | A301V + D313E + A324V + | |
| | L439V + Q441E + A537G + | |
| | N607K | |
| 270 | T72A + Q229H + V257I + | M12-3 |
| | A301V + D313E + A324V + | |
| | Q441E + A537G + N607K | |
| 271 | T72A + A137S + Q229P + | M21-18 |
| | A301V + L439V + Q441E + | |
| | A537G + N607K | |
| 272 | T72A + A137S + Q229L + | M21-22 |
| | A301V + L439V + Q441E + | |
| | A537G + N607K | |
| 273 | T72A + A137S + Q229G + | M21-25 |
| | A301V + L439V + Q441E + | |
| | A537G + N607K | |
| 274 | T72A + Q229I + V257I + | M22-25 |
| | A301V + D313E + A324V + | |
| | L439V + Q441E + A537G + | |
| | N607K | |
| 275 | T72A + A137S + I228G + | M24-1 |
| | Q229P + A301V + L439V + | |
| | Q441E + A537G + N607K | |
| 276 | T72A + A137S + I228L + | M24-2 |
| | Q229P + A301V + L439V + | |
| | Q441E + A537G + N607K | |
| 277 | T72A + A137S + I228D + | M24-5 |
| | Q229P + A301V + L439V + | |
| | Q441E + A537G + N607K | |
| 278 | T72A + A137S + Q229P + | M26-3 |
| | I230D + A301V + L439V + | |
| | Q441E + A537G + N607K | |
| 279 | T72A + A137S + Q229P + | M26-5 |
| | I230V + A301V + L439V + | |
| | Q441E + A537G + N607K | |
| 280 | T72A + I228S + Q229H + | M29-3 |
| | V257I + A301V + D313E + | |
| | A324V + L439V + Q441E + | |
| | A537G + N607K | |
| 281 | T72A + Q229H + S256C + | M33-1 |
| | V257I + A301V + D313E + | |
| | A324V + L439V + Q441E + | |
| | A537G + N607K | |
| 282 | T72A + A137S + Q229P + | M35-4 |
| | V257I + A301V + A324V + | |
| | L439V + Q441E + A537G + | |
| | N607K | |
| 283 | T72A + A137S + Q229P + | M37-5 |
| | A301V + A324V + L439V + | |
| | Q441E + A537G + N607K | |
| 284 | T72A + Q229P + V257I + | M39-4 |
| | A301G + D313E + A324V + | |
| | Q441E + A537G + N607K | |
| 285 | T72A + Q229P + V257I + | M41-2 |
| | A301V + D313E + A324V + | |
| | Q441E + A537G + N607K | |
| 286 | T72A + A137S + V184A + | M35-4/V184A |
| | Q229P + V257I + A301V + | |
| | A324V + L439V + Q441E + | |
| | A537G + N607K | |
| 287 | T72A + A137S + V184G + | M35-4/V184G |
| | Q229P + V257I + A301V + | |
| | A324V + L439V + Q441E + | |
| | A537G + N607K | |
| 288 | T72A + A137S + V184N + | M35-4/V184N |
| | Q229P + V257I + A301V + | |
| | A324V + L439V + Q441E + | |
| | A537G + N607K | |
| 289 | T72A + A137S + V184S + | M35-4/V184S |
| | Q229P + V257I + A301V + | |
| | A324V + L439V + Q441E + | |
| | A537G + N607K | |
| 290 | T72A + A137S + V184T + | M35-4/V184T |
| | Q229P + V257I + A301V + | |
| | A324V + L439V + Q441E + | |
| | A537G + N607K | |

The mutant protein of the present invention has an excellent peptide-synthesizing activity. That is, the mutant protein has improved performance as to capability to catalyze a peptide synthesis reaction than that of the wild type protein having the amino acid sequence of SEQ ID NO:2. More specifically, each mutant protein of the present invention has enhanced performance as to any of the properties such as a reaction rate, yield, substrate specificity, pH property and temperature stability that are required for the peptide synthesis reaction upon synthesizing a peptide from a certain carboxy component and amine component, when compared with the wild protein (specifically, see Examples below). Thus, the mutant proteins of the invention can be used suitably for production of the peptide on an industrial scale. A preferable embodiment of the mutant protein may be those having the ability to achieve preferably 1.3 times or more, more preferably 1.5 times or more and still more preferably 2 times or more peptide concentration when the peptide concentration achieved by the wild type protein is “1”.

In the present specification, the peptide-synthesizing activity refers to an activity which forms a peptide bond from two or more substances to synthesize a new compound having the peptide bond. As a specific example, the peptide-synthesizing activity may refer to an activity to synthesize from two amino acids or esters thereof a peptide having another one peptide bond.

The mutation shown in the mutations 1 to 38 may be introduced by modifying the nucleotide sequence so that the amino acid at a specific position is substituted in the gene encoding the protein having the amino acid sequence of SEQ ID NO:2 by, for example a site-directed mutagenesis method. The nucleotide sequence corresponding to a mutation position in the amino acid sequence of SEQ ID NO:2 may be identified easily with reference to SEQ ID NO:1. A polypeptide having the modified nucleotide sequence may be obtained by conventional mutagenesis. Examples of the mutagenesis may include a method of treatment of a DNA encoding a protein (A) with hydroxylamine in vitro, a method of introduction of the mutation by error-prone PCR, and a method of amplification of the DNA in a host which lacks a mutation repair system and subsequent retrieval of the mutated DNA.

Substantially the same mutant protein as the mutant protein containing one or more mutations shown in the above mutations 1 to 38 is also provided in accordance with the present invention. That is, the present invention also provides the mutant protein having an amino acid sequence further containing one or several amino acid mutations selected from the group consisting of substitution, deletion, insertion, addition and inversion at positions other than one or more mutation positions of the above mutations 1 to 38 in the mutant protein containing one or more mutations of the mutations 1 to 38, and having the peptide-synthesizing activity (the protein may be referred to hereinbelow as the “mutant protein (II)”). That is, the mutant protein of the present invention may contain the mutation at the position other than the positions of the mutations 1 to 38 in the amino acid shown in SEQ ID NO:2. Therefore, when the mutation such as deletion and insertion is introduced to the position other than the mutation positions of the mutations 1 to 38, the number of the amino acids from the position specified by the mutations 1 to 38 to the N terminus or the C terminus may be different from the number before introducing the mutation.

As used herein, “several amino acids” may vary depending on positions and types of amino acid residues in the three dimensional structure of the protein, but may be in a range so as not to significantly impair the three dimensional structure of the protein with the amino acid residues. Specifically, “several” may refer to 2 to 50, preferably 2 to 30, and more preferably 2 to 10 amino acids. In the case of the mutant protein containing the mutation other than the mutations 1 to 38, it is desirable to retain about a half or more, more preferably 80% or more, still more preferably 90% or more and in particular preferably 95% or more activity of that of the peptide-synthesizing activity in the protein containing one or more mutations of the mutations 1 to 38 (i.e., the mutant protein (I)) under the condition of 50° C. and pH 8.

The mutation other than the mutations 1 to 38 may also be obtained by the site-directed mutagenesis method for modifying the nucleotide sequence so that an amino acid at a specific position in a gene encoding the present protein is substituted, deleted, inserted, or added. A polypeptide having the modified nucleotide sequence may also be obtained by the conventional mutagenesis. Examples of the mutagenesis may include a method of treating a DNA encoding the mutant protein (I) with hydroxylamine in vitro and a method treating a microorganism belonging to genus Escherichia holding the DNA encoding the mutant protein (I) with ultraviolet ray, or a conventional mutagen for artificial mutagenesis such as N-methyl-N′-nitro-N-nitrosoguanidine (NTG) and nitrous acid.

The mutations such as substitution, deletion, insertion addition and inversion of nucleotides as described above encompass naturally occurring mutations such as differences owing to species or strains of the microorganisms. A DNA encoding substantially the same protein as the protein described in SEQ ID NO:2 may be obtained by expressing a DNA having the mutation as the above in an appropriate cell and examining the enzyme activity among the expressed products.

2. Polynucleotide of the Present Invention

The present invention provides a polynucleotide encoding the amino acid sequence of the mutant protein of the present invention. Depending on codon degeneracy, there can be a plurality of nucleotide sequences which define one amino acid sequence. That is, the polynucleotide of the present invention includes the following polynucleotides.

(i) The polynucleotide encoding the mutant protein having the amino acid sequence containing one or more mutations selected from the aforementioned mutations 1 to 38 in the amino acid sequence described in SEQ ID NO:2.

(ii) The polynucleotide encoding the mutant protein having the amino acid sequence further containing one or several amino acid mutations selected from the group consisting of substitution, deletion, insertion, addition and inversion at positions other than one or more mutation positions of the mutations 1 to 38 in the mutant protein described in above (I), and having the peptide-synthesizing activity.

The amino acid sequence of SEQ ID NO:2 is encoded by, for example, the nucleotide sequence described in SEQ ID NO:1.

A polynucleotide which is substantially the same as the DNA having the nucleotide sequence shown in SEQ ID NO:1 may include the following polynucleotides. The specific polynucleotide to be separated may be a polynucleotide composed of a nucleotide sequence which hybridizes under a stringent condition with a polynucleotide complementary to the nucleotide sequence described in SEQ ID NO:1, or a probe prepared from the nucleotide sequence; and encodes a protein having the peptide-synthesizing activity. The specific polynucleotide may be isolated from the polynucleotide encoding the protein having the amino acid sequence described in SEQ ID NO:2 or from cells keeping the same. The polynucleotide which is substantially the same as the polynucleotide having the nucleotide sequence described in SEQ ID NO:1 may thus be obtained.

The present invention also provides a polynucleotide shown in the following (iii) or (iv) which is substantially the same as the polynucleotide encoding the mutant protein of the invention.

(iii) The polynucleotide which hybridizes with polynucleotide having the nucleotide sequence complementary to the nucleotide sequence of the polynucleotide in the above (i) under the stringent condition, and encodes the protein keeping one or more mutations of the mutations 1 to 38 and having the peptide-synthesizing activity.

(iv) The polynucleotide which hybridizes with polynucleotide having the nucleotide sequence complementary to the nucleotide sequence of the polynucleotide in the above (ii) under the stringent condition, and encodes the protein keeping one or more mutations of the mutations 1 to 38 and having the peptide synthesizing activity.

A probe for obtaining substantially the same polynucleotide may be made by standard methods based on the nucleotide sequence described in SEQ ID NO:1 or the nucleotide sequence encoding the mutant protein. Also, using the probe, a polynucleotide which hybridizes therewith may be picked up and the objective polynucleotide may be isolated by the standard methods. For example, a DNA probe may be prepared by amplifying the nucleotide sequence that has been cloned into a plasmid or a phage vector, cutting out the nucleotide sequence to be used as the probe, and extracting it. A site to be cut out may be adjusted depending on the objective DNA.

As used herein, the “stringent condition” refers to the condition where a so-called specific hybrid is formed whereas non-specific hybrid is not formed. Although it is difficult to clearly quantify this condition, examples thereof may include the condition where a pair of DNA sequences with high homology, e.g., DNA sequences having the homology of 50% or more, more preferably 80% or more, still more preferably 90% or more and in particular preferably 95% or more are hybridized whereas DNA with lower homology than that are not hybridized, or a washing condition of an ordinary Southern hybridization, i.e., hybridization at salt concentrations equivalent to 1×SSC and 0.1% SDS at 60° C. and preferably 0.1×SSC and 0.1% SDS at 60° C. Genes which has hybridized under such a condition may include those where a stop codon has occurred in the sequence and the activity has been lost because of the mutated active center, but these may be easily removed by linking to a commercially available vector, expressing in an appropriate host and determining an enzyme activity of an expressed product by methods described later.

As described above, in the cases of the polynucleotides in the above (ii), (iii) and (iv), it is desirable that the proteins encoded thereby retain about a half or more, more preferably 80% or more and still more preferably 90% or more activity of the peptide-synthesizing activity in the mutant protein in the above (I).

3. Protein Having Amino Acid Sequence Described in SEQ ID NO:2

As described above, the mutant protein (I) may be obtained by modifying the protein having the amino acid sequence described in SEQ ID NO:2. The protein which was used as a source of the protein of the present invention will be described below. The mutant protein of the invention is not limited to an origin of the protein.

The DNA described in SEQ ID NO:1 and the protein having the amino acid sequence described in SEQ ID NO:2 are derived from Sphingobacterium multivorum FERM BP-10163 strain (indication given by the depositor for identification: Sphingobacterium multivorum AJ 2458). Microbial strains having an FERM number have been deposited to International Patent Organism Depositary, National Institute of Advanced Industrial Science and Technology, (Central No. 6, 1-1-1 Higashi, Tsukuba, Ibaraki Prefecture, Japan), and can be furnished with reference to the accession number.

A homogeneous protein to the protein having the amino acid sequence described in SEQ ID NO:2 may be isolated from Sphingobacterium sp. FERM BP-8124 strain. The protein where leucine, the amino acid residue at position 439 in the protein having the amino acid sequence described in SEQ ID NO:2 has been substituted with valine is isolated from
Sphingobacterium sp. FERM BP-8124 strain. Sphingobacterium sp. FERM BP-8124 strain (indication given by the depositor for identification: Sphingobacterium sp. AJ 110003) was deposited on Jul. 22, 2002 to International Patent Organism Depositary, National Institute of Advanced Industrial Science and Technology, and the accession number was given. Microbial strains having the FERM number have been deposited to International Patent Organism Depositary, National Institute of Advanced Industrial Science and Technology, (Central No. 6, 1-1-1 Higashi, Tsukuba, Ibaraki Prefecture, Japan), and can be furnished with reference to the accession number.

The aforementioned microbial strain of Sphingobacterium multivorum was identified to be of Sphingobacterium multivorum by the following classification experiments. The aforementioned microbial strain had the following natures: bacillus (0.6 to 0.7×1.2 to 1.5 μm), gram negative, no sporogenesis, no mobility, circular colony form, smooth entire fringe, low convex, lustrous shining, yellow color, grown at 30° C., catalase positive, oxidase positive and OF test (glucose) negative, and was thereby identified to be of genus Sphingobacterium. Furthermore, the microbial strain was proven to be similar to Sphingobacterium multivorum in characterization by the following natures: nitrate reduction negative, indole production negative, negative for acid generation from glucose, arginine dihydrase negative, urease positive, aesculin hydrolysis positive, gelatin hydrolysis negative, β-galactosidase positive, glucose utilization positive, L-arabinose utilization positive, D-mannose utilization positive, D-mannitol utilization negative, N-acetyl-D-glucosamine utilization positive, maltose utilization positive, potassium gluconate utilization negative, n-capric acid utilization negative, adipic acid utilization negative, dl-malic acid utilization negative, sodium citrate utilization negative, phenyl acetate utilization negative and cytochrome oxidase positive. In addition, as a result of a homology analysis of a nucleotide sequence of 16S rRNA gene, the highest homology (98.5%) to Sphingobacterium multivorum was exhibited, and thus, the present microbial strain was identified as Sphingobacterium multivorrum.

A DNA consisting of a nucleotide sequence of the base numbers 61 to 1917 in SEQ ID NO:1 is a code sequence portion. The nucleotide sequence of the base numbers 61 to 1917 includes a signal sequence region and a mature protein region. The signal sequence region is the region of the base numbers 61 to 120, and the mature protein region is the region of the base numbers 121 to 1917. That is, the present invention provides both a peptide enzyme protein gene containing the signal sequence and a peptide enzyme protein gene as the mature protein. The signal sequence containing the sequence described in SEQ ID NO:11 is a class of a leader sequence, and a major function of a leader peptide encoded in the leader sequence region is presumed to be secretion thereof from a cell membrane inside to a cell membrane outside. The protein encoded by the nucleotide sequence of the base numbers 121 to 1917, i.e., the region except the leader peptide sequence corresponds to the mature protein, and is presumed to have the high peptide-synthesizing activity.

The DNA having the nucleotide sequence of SEQ ID NO:1 may be obtained from a chromosomal DNA of Sphingobacterium multivorum or a DNA library by PCR (polymerase chain reaction, see White, T. J. et al ;Trends Genet., 5, 185(1989)) or hybridization. Primers for PCR may be designed based on an internal amino acid sequence determined on the basis of the purified protein having the peptide-synthesizing activity. The primer or a probe for the hybridization may be designed based on the nucleotide sequence described in SEQ ID NO:1, or may also be isolated using a probe. When the primers having the sequences corresponding to a 5′-untranslated region and a 3′-untranslated region as the PCR primers, a full length coding region of the present protein may be amplified. Explaining as an example the primers for amplifying the region including the region encoding both the leader sequence and the mature protein described in SEQ ID NO:1, a primer having the nucleotide sequence of the upstream of the base number 61 in SEQ ID NO:1 may be used as the 5′-primer, and a primer having a sequence complementary to the nucleotide sequence of the downstream of the base number 1917 may be used as the 3′-primer.

The primers may be synthesized in accordance with standard methods, for example, by a phosphoamidite method (see Tetrahedron Letters, 22:1859, 1981) using a DNA synthesizer model 380B supplied from Applied Biosystems. The PCR reaction may be performed, for example, using Gene Amp PCR System 9600 (supplied from Perkin Elmer) and TaKaRa LA PCR in vitro Cloning Lit (supplied from Takara Shuzo Co., Ltd.) in accordance with instructions from the supplier such as manufacturer.

4. Method for Producing Mutant Protein of the Present Invention

The method for producing the protein of the present invention and the methods for producing recombinants and transformants used therefor will be subsequently described.

A transformant which expresses the aforementioned mutant protein can produce the mutant protein having the peptide-synthesizing activity. For example, the mutant protein having the activity may be produced by introducing the mutation corresponding to any of the mutations 1 to 38 into a recombinant DNA such as an expression vector having the nucleotide sequence shown in SEQ ID NO:1, and introducing the expression vector into an appropriate host to express the mutant protein. As the host for expressing the mutant protein specified by the DNA having the nucleotide sequence of SEQ ID NO:1, it is possible to use various prokaryotic cells such as microorganisms belonging genera Escherichia (e.g., Escherichia coli), Empedobacter, Sphingobacterium and Flavobacterium, and Bacillus subtilis as well as various eukaryotic cells such as Saccharomyces cerevisiae, Pichia stipitis, and Aspergillus oryzae.

The recombinant DNA for introducing a foreign DNA into the host may be prepared by inserting a predetermined DNA into the vector selected depending on the type of the host in a manner whereby a protein encoded by the DNA can be expressed. When a promoter inherent for a gene encoding the protein produced by Empedobacter brevis works in the host cell, that promoter may be used as the promoter for expressing the protein. If necessary, another promoter which works in the host cell may be ligated to the DNA encoding the mutant protein, which may be then expressed under the control of that promoter.

Examples of a transformation method for introducing the recombinant DNA into the host cell may include D. M. Morrison's method (Methods in Enzymology 68, 326 (1979)) or a method of enhancing permeability of the DNA by treating recipient microorganisms with calcium chloride (Mandel, M. and Higa, A., J. Mol. Biol., 53, 159 (1970)).

In the case of producing a protein on a large scale using the recombinant DNA technology, one of the preferable embodiments therefor may be formation of an inclusion body of the protein. The inclusion body is configured by aggregation of the protein in the protein-producing transformant. The advantages of this expression production method may be protection of the objective protein from digestion by protease which is present in the microbial cells, and ready purification of the objective protein that may be performed by disruption of the microbial cells and following centrifugation.

The protein inclusion body obtained in this way may be solubilized by a protein denaturing agent, which is then subjected to activation regeneration mainly by removing the denaturing agent, to be converted into correctly refolded and physiologically active protein. There are many examples of such procedures, such as activity regeneration of human interleukin 2 (JP-S61-257931 A).

To obtain the active protein from the protein inclusion body, a series of the manipulations such as solubilization and activity regeneration is required, and thus, the manipulations are more complicate than those in the case of directly producing the active protein. However, when a protein which affects microbial cell growth is produced on a large scale in the microbial cells, effects thereof may be inhibited by accumulating the protein as the inactive inclusion body in the microbial cells.

Examples of the methods for producing the objective protein on a large scale as the inclusion body may include methods of expressing the protein alone under control of a strong promoter, as well as methods of expressing the objective protein as a fusion protein with a protein known to be expressed in a large amount.

As an example, a method for preparing transformed Escherichia coli and producing a mutant protein using this will be described more specifically hereinbelow. When the mutant protein is produced by microorganisms such as E. coli, a DNA encoding a precursor protein including the leader sequence may be incorporated or a DNA for a mature protein region without including the leader sequence may be incorporated as a code sequence of the protein. Either one may be appropriately selected depending on the production condition, the form and the use condition of the enzyme to be produced.

As the promoter for expressing the DNA encoding the mutant protein, the promoter typically used for producing xenogenic proteins in E. coli may be used, and examples thereof may include strong promoters such as T7 promoter, lac promoter, trp promoter, trc promoter, tac promoter, and PR promoter and PL promoter of lambda phage. As the vector, pUC19, pUC18, pBR322, pHSG299, pHSG298, pHSG399, pHSG398, RSF1010, pMW119, pMW118, pMW219, and pMW218 may be used. Other vectors of phage DNA may also be used. In addition, expression vectors which contains a promoter and can express the inserted DNA sequence may also be used.

In order to produce the mutant protein as a fusion protein inclusion body, a fusion protein gene is made by linking a gene encoding another protein, preferably a hydrophilic peptide to upstream or downstream of the mutant protein gene. Such a gene encoding the other protein may be those which increase an amount of the accumulated fusion protein and enhance solubility of the fusion protein after denaturation and regeneration steps. Examples of candidates thereof may include T7 gene 10, β-galactosidase gene, dehydrofolic acid reductase gene, interferon γ gene, interleukin-2 gene and prochymosin gene.

Such a gene may be ligated to the gene encoding the mutant protein so that reading frames of codons are matched. This may be effected by ligating at an appropriate restriction enzyme site or using a synthetic DNA having an appropriate sequence.

In some cases, it is preferable to ligate a terminator, i.e. the transcription termination sequence, to downstream of the fusion protein in order to increase the production amount. Examples of this terminator may include T7 terminator, fd phage terminator, T4 terminator, tetracycline resistant gene terminator and E. coli trpA gene terminator.

The vector for introducing the gene encoding the mutant protein or the fusion protein of the mutant protein with the other protein into E. coli may preferably be of a so-called multicopy type. Examples thereof may include plasmids having a replication origin derived from ColE1 , such as pUC based plasmids, pBR322 based plasmids or derivatives thereof. As used herein, the “derivative” means the plasmid modified by the substitution, deletion, insertion, addition and/or inversion of a base(s). “Modified” referred to herein includes the modification by mutagenesis with the mutagen or UV irradiation and natural mutation.

In order to select the transformants, it is preferable that the vector has a marker such as an ampicillin resistant gene. As such a plasmid, expression vectors having the strong promoter are commercially available (pUC series: Takara Shuzo Co., Ltd., pPROK series and pKK233-2: Clontech, etc.).

A DNA fragment where the promoter, the gene encoding the protein having the peptide-synthesizing activity or the fusion protein of the protein having the peptide-synthesizing activity with the other protein, and in some cases the terminator are ligeted sequentially is then ligeted to the vector DNA to obtain a recombinant DNA.

The mutated protein or the fusion protein of the mutated protein with the other protein is expressed and produced by transforming E. coli with the resulting recombinant DNA and culturing this E. coli. Strains commonly used for the expression of the xenogenic gene may be used as the host to be transformed. E. coli JM 109 strain which is a subspecies of E. coli K12 strain is preferable. The methods for transformation and for selecting transformants are described in Molecular Cloning, 2nd edition, Cold Spring Harbor press, 1989.

In the case of expressing as the fusion protein, the fusion protein may be composed so as to be able to cleave the peptide-synthesizing enzyme therefrom using a restriction protease which recognizes a sequence of blood coagulation factor Xa, kallikrein or the like which is not present in the peptide-synthesizing enzyme.

As production media, the media such as M9-casamino acid medium and LB medium typically used for cultivation of E. coli may be used. The conditions for cultivation and a production induction may be appropriately selected depending on types of the marker and the promoter of the vector and the host used.

The following methods are available for recovering the mutant protein or the fusion protein of the mutant protein with the other protein. If the mutant protein or the fusion protein thereof is solubilized in the microbial cells, the cells may be collected and then disrupted or lysed to thereby obtain a crude enzyme solution. If necessary, the crude solution may be purified using techniques such as ordinary precipitation, filtration and column chromatography, to obtain purified mutant protein or the fusion protein. In this case, the purification may be performed using an antibody against the mutant protein or the fusion protein.

In the case where the protein inclusion body is formed, this may be solubilized with a denaturing agent. The inclusion body may be solubilized together with the microbial cells. However, considering the following purification process, it is preferable to take up the inclusion body before solubilization. Collection of the inclusion body from the microbial cells may be performed in accordance with conventionally and publicly known methods. For example, the microbial cells are disrupted, and the inclusion body is then collected by centrifugation and the like. Examples of the denaturing agent which solubilizes the protein inclusion body may include guanidine-hydrochloric acid (e.g., 6 M, pH 5 to 8), urea (e.g., 8 M), and the like.

As a result of removal of the denaturing agent by dialysis and the like, the protein may be regenerated as the protein having the activity. Dialysis solutions used for the dialysis may include Tris hydrochloric acid buffer, phosphate buffer and the like. The concentration thereof may be 20 mM to 0.5 M, and pH thereof may be 5 to 8.

It is preferred that the protein concentration at a regeneration step is kept at about 500 μg/ml or less. In order to inhibit self-crosslinking of the regenerated peptide-synthesizing enzyme, it is preferred that dialysis temperature is kept at 5° C. or below. Methods for removing the denaturing agent other than the dialysis method may include a dilution method and an ultrafiltration method. The regeneration of the activity is anticipated by using any of these methods.

5. Method for Producing Peptide

In the method for producing the peptide of the present invention, a peptide is synthesized using the aforementioned mutant protein. That is, in the method for producing the peptide of the present invention, an amine component and a carboxy component are reacted to synthesize a peptide in the presence of at least one of the following proteins (I) and (II).

(I) The mutant protein having the amino acid sequence containing one or more mutations selected from the aforementioned mutations 1 to 38 in the amino acid sequence described in SEQ ID NO:2.

(II) The mutant protein having the amino acid sequence containing one or several mutations selected from the group consisting of substitution, deletion, insertion, addition and inversion at positions other than one or more mutations selected from any of the mutations 1 to 38 in the mutant protein described in the above (I), and having the peptide-synthesizing activity.

In the method for producing the peptide of the present invention, the mutant protein is placed in the peptide-synthesizing reaction system. The mutant protein may be supplied as a mixture containing the protein (I) and/or (II) in a biochemically acceptable solvent (the mixture will be referred to hereinbelow as “mutant protein-containing material”). More specifically, the peptide may be synthesized from the amine component and the carboxy component using one or more selected from the group consisting of a cultured product of a microorganism that has been transformed so as to express the mutant protein of the present invention, a microbial cell separated from the cultured product and the treated microbial cells of the microorganism.

As used herein, the “mutant protein-containing material” may be any material containing the mutant protein of the present invention, and specifically includes a cultured product of microorganisms which produce the mutant protein, microbial cells separated from the cultured product, and the treated microbial cells. The cultured product of microorganisms refers to one obtained by cultivation of the microorganisms, and more specifically refers to, e.g., a mixture of microbial cells, the medium used for culturing the microorganisms and substances produced by the cultured microorganisms. Alternatively, the microbial cells may be washed, and used as the washed microbial cells. The treated microbial cells may include ones obtained by disrupting, lysing and lyophilizing the microbial cells, as well as crude purified proteins recovered by further treating the microbial cells, and purified proteins obtained by further purification. As the purified proteins, partially purified proteins obtained by various purification methods may be used, and immobilized proteins obtained by immobilizing by a covalent bond method, an absorption method or an entrapment method may also be used. Depending on the microorganism to be used, bacteriolysis may partially occurs during the cultivation. In this case, a cultured supernatant may also be used as the mutant protein-containing material.

As the microorganism containing the mutant protein of the present invention, a gene recombinant strain which expresses the mutant protein may be used. Alternatively, treated microbial cells such as microbial cells treated with acetone and lyophilized microbial cells may be used. These may further be immobilized by a variety of methods such as the covalent bond method, the absorption method or the entrapment method, to produce immobilized microbial cells or immobilized treated microbial cells for use.

When the cultured product, the cultured microbial cells, the washed microbial cells and the treated microbial cells such as disrupted or lysed microbial cells are used, these materials tend to contain enzymes which are not involved in peptide production and degrade produced peptides. In this case, it is sometimes preferable to add a metal protease inhibitor such as ethylenediamine tetraacetatic acid (EDTA). The amount of such an inhibitor to be added may be in the range of 0.1 mM to 300 mM, and preferably from 1 mM to 100 mM.

The mutant protein or the mutant protein-containing material may be allowed to act upon a carboxy component and an amine component merely by mixing the mutant protein or the mutant protein-containing material, the carboxy component and the amine component. More specifically, the mutant protein or the mutant protein-containing material may be added to a solution containing the carboxy component and the amine component to react. Alternatively, in the case of using microorganisms which produce the mutant protein, the microorganisms which produce the mutant protein may be cultured to generate and accumulate the enzyme in the microorganisms or a cultured medium of the microorganisms, and the carboxy component and the amine component may then be added into the cultured medium. The produced peptide may be recovered in accordance with standard methods, and purified as needed.

To obtain microbial cells (cells of the microorganisms), the microorganisms may be cultured and grown in an appropriate cultivation medium which may be selected depending on the type of the microorganisms. The medium therefor is not particularly limited as long as the microorganisms can be grown in the medium, and may be an ordinary medium containing carbon sources, nitrogen sources, phosphorus sources, sulfur sources, inorganic ions, and, if necessary, organic nutrient sources.

Any carbon sources may be used as long as the microorganism can utilize. Examples of the carbon sources may include sugars such as glucose, fructose, maltose and amylose, alcohols such as sorbitol, ethanol and glycerol, organic acids such as fumaric acid, citric acid, acetic acid and propionic acid and salts thereof, carbohydrates such as paraffin, and mixtures thereof.

As the nitrogen sources, ammonium salts of inorganic acids such as ammonium sulfate and ammonium chloride, ammonium salts of organic acids such as ammonium fumarate and ammonium citrate, nitrate salts such as sodium nitrate and potassium nitrate, organic nitrogen compounds such as peptone, yeast extract, meat extract and corn steep liquor, or mixtures thereof may be used.

If necessary, nutrient sources such as inorganic salts, trace metal salts and vitamins commonly used in the medium may be admixed for use.

A cultivation condition is not particularly limited, and the cultivation may be performed under an aerobic condition at pH 5 to 9 and at a temperature ranging from about 15 to 55° C. for about 12 to 48 hours while appropriately controlling pH and the temperature.

A preferable embodiment of the method for producing the peptide of the present invention may be a method in which the transformed microorganisms are cultured in the medium to accumulate the peptide-synthesizing enzyme in the medium and/or the microorganisms. Employment of the transformants enables production of the mutant protein readily on a large scale, and thus the peptide may thereby be rapidly synthesized in a large amount.

The amount of the mutant protein or the mutant protein-containing material to be used may be the amount by which an objective effect is exerted (i.e., effective amount). Those skilled in the art can easily determine this effective amount by a simple preliminary experiment. For example, the effective amount is about 0.01 to 100 units (U) or about 0.1 to 500 g/L in the case of using the enzyme or the washed microbial cells, respectively.

Any carboxy component may be used as long as it can be condensed with the amine component, the other substrate, to generate the peptide. Examples of the carboxy component may include L-amino acid ester, D-amino acid ester, L-amino acid amide, D-amino acid amide, and organic acid ester having no amino group. As amino acid ester, not only amino acid esters corresponding to natural amino acids but also amino acid esters corresponding to non-natural amino acids and derivatives thereof are also exemplified. In addition, as amino acid esters, β-, γ-, and ω-amino acid esters in addition to α-amino acid ester having different binding sites of amino groups are also exemplified. Representative examples of amino acid esters may include methyl ester, ethyl ester, n-propyl ester, iso-propyl ester, n-butyl ester, iso-butyl ester and tert-butyl ester of amino acids.

Any amine component may be used as long as it can be condensed with the carboxy component, the other substrate, to generate the peptide. Examples of the amine component may include L-amino acid, C-protected L-amino acid, D-amino acid, C-protected D-amino acid and amines. As amines, not only natural amine but also non-natural amine and derivatives thereof are exemplified. As amino acids, not only natural amino acids but also non-natural amino acids and derivatives thereof are exemplified. β-, γ-, and ω-Amino acids in addition to a-amino acids having different binding sites of amino groups are also exemplified.

Concentrations of the carboxy component and the amine component which are starting materials may be 1 mM to 10 M and preferably 0.05 M to 2 M. In some cases, it is preferable to add the amine component in the amount equal to or more than the amount of the carboxy component. When the reaction is inhibited by the high concentration of the substrate, the concentrations may be kept to a certain level in order to avoid inhibition of the reaction and the components may be sequentially added.

A reaction temperature may be 0 to 60° C. at which the peptide can be synthesized, and preferably 5 to 40° C. A reaction pH may be 6.5 to 10.5 at which the peptide can be synthesized, and preferably pH 7.0 to 10.0.

The method for producing the peptide of the present invention is suitable for methods for producing various peptides. The method for producing the peptide of the present invention is also suitable for the method for producing, for example, α-L-aspartyl-L-phenylalanine-β-methyl ester (i.e., α-L-(β-O-methyl aspartyl)-L-phenylalanine, abbreviated as α-AMP). α-AMP is an important intermediate for producing α-L-aspartyl-L-phenylalanine-α-methyl ester (product name: Aspartame) which has a large demand as a sweetener.

### EXAMPLES

The present invention will be described in detail with reference to the following Examples, but the invention is not limited thereto.

### Example 1

### Expression of Peptide-Synthesizing Enzyme Gene in E. coli

An objective gene encoding a protein having a peptide-synthesizing activity was amplified by PCR with a chromosomal DNA from Sphingobacterium multivorum FERM BP-10163 strain as a template using oligonucleotides shown in SEQ ID NOS:5 and 6 as primers. An amplified DNA fragment was treated with NdeI/XbaI, and a resulting DNA fragment was ligated to pTrpT that had been treated with NdeI/XbaI. Escherichia coli JM109 was transformed with this solution containing the ligated product, and a strain having an objective plasmid was selected with ampicillin resistance as an indicator, and this plasmid was designated as pTrpT_Sm_Aet. Escherichia coli JM109 having pTrpT_Sm_Aet is also represented as pTrpT_Sm_Aet/JM109 strain.

One platinum loopful of pTrpT_Sm_Aet/JM109 strain was inoculated into a general test tube in which 3 mL of a medium (2 g/L of glucose, 10 g/L of yeast extract, 10 g/L of casamino acid, 5 g/L of ammonium sulfate, 3 g/L of potassium dihydrogen phosphate, 1 g/L of dipotassium hydrogen phosphate, 0.5 g/L of magnesium sulfate 7-hydrate, 100 mg/L of ampicillin) had been placed, and a main cultivation was performed at 25° C. for 20 hours. An L-alaninyl-L-glutamine-synthesizing activity of 2.1 U per 1 mL of the cultured medium was found, thereby confirming that the cloned gene had been expressed in Escherichia coli. No activity was detected in transformants into which pTrpT alone had been introduced as a control.

### Example 2

### Construction of Rational Mutant Strain Using pKF Vector

(1) Construction of pKF_Sm_Aet

An objective gene was amplified by PCR with pTrpT_Sm_Aet plasmid as a template using the oligonucleotides shown in SEQ ID NOS:3 and 4 as the primers. This DNA fragment was treated with EcoRI/PstI, and the resulting DNA fragment was ligated to pKF18k2 (supplied from Takara Shuzo Co., Ltd.) that had been treated with EcoRI/PstI. Escherichia coli JM109 was transformed with this solution containing the ligated product, and a strain having an objective plasmid was selected with kanamycin resistance as the indicator, and this plasmid was designated as pKF_Sm_Aet. Escherichia coli JM109 having pKF_Sm_Aet is also represented as pKF_Sm_Aet/JM109 strain.

(2) Introduction of Rational Mutation into pKF-Sm_Aet

In order to construct mutant Aet, pKF_Sm_Aet plasmid was used as the template for site-directed mutagenesis using an ODA method. Mutations were introduced using “site-directed mutagenesis system Mutan Super Express kit” supplied from Takara Shuzo Co., Ltd. (Japan) in accordance with the protocol of the manufacturer using the primers (SEQ ID NOS:12 to 33) corresponding to each mutant enzyme. The 5′ terminus of the primers were phosphorylated before use with T4 polynucleotide kinase supplied from Takara Shuzo Co., Ltd. The primers were phosphorylated by adding 100 μmol DNA (primer) and 10 units of T4 polynucleotide kinase to 20 μL of 50 mM tris-hydrochloric acid buffer (pH 8.0) containing 0.5 mM ATP, 10 mM magnesium chloride and 5 mM DTT and warming at 37° C. for 30 minutes followed by heating at 70° C. for 5 minutes. Subsequently, 1 μL (5 pmol) of this reaction solution was used for PCR by which the mutation was introduced. The PCR was performed by adding 10 ng of ds DNA (pKF_Sm_Aet plasmid) as the template, 5 pmol each of Selection Primer and 5′-phosphorylated mutagenic oligonucleotides shown above as the primers and 40 units of LA-Taq to 50 μL of LA-Taq buffer II (Mg²⁺ plus) containing 250 μM each of dATP, dCTP, dGTP and dTTP, which was then subjected to 25 cycles of heating at 94° C. for one minute, 55° C. for one minute and 72° C. for 3 minutes. After the PCR for introducing the mutation was completed, a DNA fragment was collected by ethanol precipitation, and Escherichia coli MV1184 strain was transformed with the resulting DNA fragment. A strain having an objective plasmid: pKF_Sm_AetM containing a mutant Aet gene was selected with kanamycin resistance as the indicator.

In the present specification, Escherichia coli MV1184 strain having pKF_Sm_AetM is also represented as pKF_Sm_AetM/MV1184 strain. When referring to a specific mutant of pKF_Sm_AetM, the mutation thereof may be represented by replacing “AetM” with the type of mutation, e.g., pKF_Sm_F207V. When a mutant contains two or more mutations, the mutations may be stated continuously with “/” dividing each mutation. For example, pKF_Sm_F207V/Q441E represents a mutant in which the mutations F207V and Q441E have been introduced into the Aet gene which pKF_Sm_Aet plasmid carries.

(3) Construction of pHSG_Sm_Aet

An objective gene was amplified by PCR with pTrpT_Sm_Aet plasmid as a template using the oligonucleotides shown in SEQ ID NO:3 and 4 as primers. This DNA fragment was treated with EcoRI/PstI, and a resulting DNA fragment was ligated to pHSG298 (suppled from Takara Shuzo Co., Ltd.) that had been treated with EcoRI/PstI. Escherichia coli MV1184 strain was transformed with this solution containing the ligated product, and a strain having an objective plasmid was selected with kanamycin resistance as an indicator, and this plasmid was designated as pHSG_Sm_Aet. Escherichia coli MV1184 having pHSG_Sm_Aet is also represented as pHSG_Sm_Aet/MV1184 strain.

(4) Obtaining Microbial Cells

Each of pKF_Sm_Aet/JM109 strain, pKF_Sm_Aet/MV1184 strain and pHSG_Sm_Aet/MV1184 strain was precultured in an LB agar medium (10 g/L of yeast extract, 10 g/L of peptone, 5 g/L of sodium chloride, 20 g/L of agar, pH 7.0) at 30° C. for 24 hours. One platinum loopful of microbial cells of each strain obtained from the above cultivation was inoculated into a general test tube in which 3 mL of the LB medium (0.1 M IPTG and 20 mg/L of kanamycin were added to the above medium from which the agar had been omitted) had been placed, and a main cultivation was performed at 25° C. at 150 reciprocatings/minute for 20 hours.

(5) Production of Peptide Using Microbial Cells <Synthesis of AMP>

400 μL of each cultured medium obtained in Example 2 (4) was centrifuged to collect the microbial cells. The collected cells were then suspended in 200 μL of 100 mM borate buffer (pH 9.0) containing 10 mM EDTA, 50 mM dimethyl aspartate and 100 mM phenylalanine, and reacted at 25° C. for 30 minutes. The concentration of α-AMP produced by the strain which expressed the wild type enzyme (such a strain will be referred to hereinbelow as the “wild strain”) in this reaction is shown in Table 2. For the dipeptide production by the strains which expressed various mutant enzymes (mutant strains), their ratios of production concentrations to those of the wild strain are shown in Table 2.

(6) Production of Peptide Using Microbial Cells <Synthesis of Ala-Gln>

100 μL of each cultured medium obtained in Example 2 (4) was centrifuged to collect the microbial cells. The collected cells were then suspended in 200 μL of 100 mM borate buffer (pH 9.0) containing 10 mM EDTA, 100 mM L-alanine methyl ester and 200 mM glutamine, and reacted at 25° C. for 30 minutes. The concentration of L-alanyl-L-glutamine (Ala-Gln) produced by the wild strain in this reaction is shown in Table 2. For the dipeptide production by the various mutant strains, the ratio of production concentration to that of the wild strain is shown in Table 2.

(7) Production of Peptide Using Microbial Cells <Synthesis of Phe-Met, Leu-Met>

800 μL of each cultured medium obtained in Example 2 (4) was centrifuged to collect the microbial cells. The collected cells were then suspended in 400 μL of 100 mM borate buffer (pH 9.0) containing 10 mM EDTA, 50 mM L-phenylalanine methyl ester hydrochloride or L-leucine methyl ester hydrochloride, and 100 mM L-methionine, and reacted at 25° C. for 20 minutes. The concentration of L-phenylalanyl-L-methionine (Phe-Met) or L-leucyl-L-methionine (Leu-Met) produced by the wild strain in this reaction is shown in Table 2. For the dipeptide synthesized by the various mutant strains, the ratio of production concentration with respect to that by the wild strain is shown in Table 2.

TABLE 2
Table 2

| | | SYNTHESIZED | | | |
|---|---|---|---|---|---|
| | | DIPEPTIDE NAME | | | |
| | | | Ala- | Phe- | Leu- |
| | | AMP | Gln | Met | Met |
| PRODUCTION AMOUNT | | 7.6 | 41 | 1.9 | 8.5 |
| OF CONTROL ENZYME | | | | | |
| DIPEPTIDE [mM] | | | | | |
| RATIO OF THE | K83A | 1.44 | 1.46 | 6.87 | 3.90 |
| SYNTHESIZED | R117A | 1.16 | 1.38 | | |
| DIPEPTIDE | D203N | 1.33 | 1.33 | 1.92 | 1.80 |
| CONCENTRATION | D203S | 1.97 | | | |
| IN VARIOUS | F207A | 1.32 | 1.21 | 3.01 | 2.76 |
| MUTANT STRAINS | F207S | 2.24 | 1.29 | 0.40 | 0.62 |
| TO THAT IN THE | F207I | 0.33 | 0.14 | 3.95 | 1.83 |
| WILD STRAIN* | F207V | 1.71 | 0.82 | 6.70 | 3.29 |
| | F207G | 1.71 | 0.82 | 0.61 | 0.81 |
| | F207T | 0.14 | 0.06 | 2.24 | 1.25 |
| | M208A | 0.14 | 0.13 | 7.06 | 1.79 |
| | S209A | 1.40 | 1.28 | 2.13 | 1.65 |
| | S209D | 1.25 | | | |
| | S209G | 0.41 | 0.83 | 1.79 | 1.25 |
| | Q441N | 1.90 | 1.68 | 0.61 | 0.55 |
| | Q441D | 1.24 | 0.83 | 0.74 | 0.65 |
| | Q441E | 1.29 | 1.51 | 3.46 | 1.55 |
| | Q441K | 1.92 | 1.71 | 2.17 | 1.23 |
| | N442K | 1.24 | 1.24 | 2.06 | 1.26 |
| | R445D | 1.26 | 1.23 | 1.15 | 1.13 |
| | R445F | 1.71 | 1.24 | | |
| | F207V/S209A | | | 3.15 | 1.79 |
| | K83A/F207V | 5.36 | 2.60 | 9.49 | 4.79 |
| | K83A/S209A | 4.77 | 4.47 | 0.16 | 0.57 |
| | K83A/Q441E | 6.86 | 4.61 | 7.12 | 4.43 |
| | F207V/Q441E | 4.93 | 2.28 | 6.52 | 3.85 |

*THIS SHOWS RATIO OF THE SYNTHESIZED DIPEPTIDE CONCENTRATION IN VARIOUS MUTANT STRAINS WHEN THE SYNTHESIZED DIPEPTIDE CONCENTRATION IN THE WILD STRAIN IS “1”

### Example 3

### Random Screening 1

(8) Preparation of pTrpT_Sm_Aet Random Library

In order to construct mutant Aet, pTrpT_Sm_Aet plasmid was used as the template for random mutagenesis using error prone PCR. The mutation was introduced using “GeneMorph PCR Mutagenesis Kit” supplied from Stratagene (USA) in accordance with the protocol of the manufacturer.

The PCR was performed using the oligonucleotides shown in SEQ ID NOS:5 and 6 as primers. That is, 500 ng of ds DNA (pTrpT_Sm_Aet or pTrpT_Sm_F207V plasmid) as the template, 125 ng each of the primers and 2.5 units of Mutazyme DNA polymerase were added to 50 μL of Mutazyme reaction buffer containing 200 μM each of dATP, dCTP, dGTP and dTTP, which was then subjected to the PCR using 30 cycles at 95° C. for 30 seconds, 52° C. for 30 seconds and 72° C. for 2 minutes.

The PCR product was treated with NdeI/XbaI, and the resulting DNA fragment was ligated to pTrpT that had been treated with NdeI/XbaI. Escherichia coli JM109 (suppled from Takara Shuzo Co., Ltd.) was transformed with this solution containing the ligated product in accordance with standard methods. This was plated on an LB agar medium containing 100 μg/mL of ampicillin to make a library into which the random mutation had been introduced.

(9) Screening From pTrpT_Sm_Aet Random Library: A

Escherichia coli JM109 strain transformed with the plasmid (pTrpT_Sm_AetM) containing each mutant Aet gene and Escherichia coli JM109 strain transformed with the plasmid containing the wild type Aet were inoculated to 150 μL (dispensed in wells of 96-well plate) of the medium containing 100 μg/mL of ampicillin (2 g/L of glucose, 10 g/L of yeast extract, 10 g/L of casamino acid, 5 g/L of ammonium sulfate, 1 g/L of potassium dihydrogen phosphate, 3 g/L of dipotassium hydrogen phosphate, 0.5 g/L of magnesium sulfate 7-hydrate, pH 7.5, 100 μg/mL of ampicillin), and cultured at 25° C. for 16 hours with shaking. The cultivation was performed with shaking at 1000 rotations/minute using a bio-shaker (M/BR-1212FP) supplied from TITEC.

(10) Primary Screening

The primary screening was performed using the cultured medium obtained in Example 3 (9). Selection was performed as follows. 200 μL of a reaction solution (pH 8.2) containing 10 mM phenol, 6 mM AP, 5 mM Asp (OMe)₂, 7.5 mM Phe, 3.6 U/mL of peroxidase, 0.16 U/mL of alcohol oxidase, 10 mM EDTA and 100 mM borate was added to 5 μL of the cultured medium, which was then reacted at 25° C. for about 20 minutes. After the reaction, an absorbance at 500 nm was measured, and an amount of released methanol was calculated. Those showing the large amount of released methanol were selected as those having the enzyme with high AMP-synthesizing activity.

(11) Obtaining Microbial Cells

One platinum loopful of the strain selected in the primary screening was precultured in the LB agar medium at 25° C. for 16 hours. One platinum loopful of each strain expressing the enzyme was inoculated to 2 mL of terrific medium (12 g/L of tryptone, 24 g/L of yeast extract, 2.3 g/L of potassium dihydrogen phosphate, 12.5 g/L of dipotassium hydrogen phosphate, 4 g/L glycerol, 100 mg/L of ampicillin) in a general test tube, and the main cultivation was performed at 25° C. at 150 reciprocatings/minute for 18 hours.

(12) Secondary Screening

25 μL of the cultured broth was suspended in 500 μL of 100 mM borate buffer (pH 8.5 or pH 9.0) containing 10 mM EDTA, 50 mM dimethyl aspartate and 75 mM phenylalanine, which was then reacted at 20° C. or 25° C. for 10 or 15 minutes to measure the amount of synthesized AMP. Among the secondary screened strains, the strains which exerted improved specific activity was analyzed as to their mutation points. As a result, the following mutation points were specified. The mutant strains comprising the mutants 4, 5, 6, 7, 8, 9, 10, 14, 15 and 16 were obtained from the library derived from the wild strain as a parent strain (template), and the mutant strains comprising the mutants 17, 18, 19 and 20 were obtained from the library derived from the F207V mutant strain as the parent strain.

(13) Production of Peptide Using Microbial Cells

The concentrations of AMP produced with the wild strain in the aforementioned reaction are shown in Table 3 (reaction time: 10 minutes), and the concentration of AMP produced with the mutant strain F207V is shown in Table 4 (reaction time: 15 minutes). For the dipeptide synthesized by each mutant strain, the ratio of the concentrations of the dipeptides synthesized by the mutant strain with respect to that by the parent strain are shown in Tables 3 and 4. Other conditions for the AMP synthesis reaction were the same as in the above Example 2 (5).

TABLE 3
Table 3

| | |
|---|---|
| SYNTHESIZED DIPEPTIDE NAME | AMP |

| REACTION pH | | 8.5 | 9.0 |
|---|---|---|---|
| PRODUCTION AMOUNT OF | | | |
| CONTROL ENZYME DIPEPTIDE [mM] | | 4.6 | 1.1 |
| RATIO OF THE SYNTHESIZED | Q441E | | 1.3 |
| DIPEPTIDE CONCENTRATION | A301V | 1.3 | 1.7 |
| IN VARIOUS MUTANT | V257I | 1.4 | 2.9 |
| STRAINS TO THAT IN THE | A537G | 1.4 | 1.8 |
| WILD STRAIN* | A324V | 1.2 | 1.4 |
| | N607K | 1.1 | 1.3 |
| | D313E | 1.3 | 1.4 |
| | Q229H | 1.3 | 1.6 |
| | T72A | 1.7 | 2.2 |
| | A137S | 1.4 | 1.5 |

*THIS SHOWS RATIO OF THE SYNTHESIZED DIPEPTIDE CONCENTRATION IN VARIOUS MUTANT STRAINS WHEN THE SYNTHESIZED DIPEPTIDE CONCENTRATION IN THE WILD STRAIN IS “1”

TABLE 4
Table 4

| | |
|---|---|
| SYNTHESIZED DIPEPTIDE NAME | AMP |

| REACTION pH | | 9.0 |
|---|---|---|
| PRODUCTION AMOUNT OF F207V ENZYME | | 2.5 |
| DIPEPTIDE [mM] | | |
| RATIO OF THE | G226S | 1.4 |
| SYNTHESIZED | W327G | 1.5 |
| DIPEPTIDE | Y339H | 1.4 |
| CONCENTRATION IN | D619E | 1.5 |
| VARIOUS MUTANT | | |
| STRAINS TO THAT IN | | |
| THE MOTHER STRAIN* | | |

*THIS SHOWS RATIO OF THE SYNTHESIZED DIPEPTIDE CONCENTRATION IN VARIOUS MUTANT STRAINS WHEN THE SYNTHESIZED DIPEPTIDE CONCENTRATION IN THE MOTHER STRAIN (MUTANT STRAIN F207V) IS “1”

### Example 4

### Evaluation of Specified Mutation Point by Introducing it into pKF

(14) Construction of Strain in Which Specified Mutation Point has been Introduced into pKF

The mutation point specified in Example 3 (12) was combined with already constructed pKF_Sm_F207V/Q441E to construct a triple mutant strain. The mutation was introduced in the same way as in Example 2 (2) using pKF_Sm_F207V/Q441E as the template and using the primers corresponding to various mutant enzymes (SEQ ID NOS:34 to 44 and 77). Resulting strains and the already constructed strains were cultured in the same way as in Example 2 (4).

(15) Production of Peptide Using Microbial Cells <AMP>

500 μL of the cultured medium obtained in Example 4 (14) was centrifuged to collect microbial cells. The collected cells were then suspended in 500 μL of 100 mM borate buffer (pH 8.5 or pH 9.0) containing 10 mM EDTA, 50 mM dimethyl aspartate and 100 mM phenylalanine, and reacted at 25° C. for 30 minutes. The concentrations of AMP synthesized with the wild strain in this reaction are shown in Table 5. For the dipeptide synthesized by various mutant strains, the ratio of the concentration of the dipeptide synthesized by the mutant strain with respect to that by the wild strain is shown in Table 5.

(16) Production of Peptide Using Microbial Cells <Ala-Gln>

100 μL of the cultured medium obtained in Example 4 (14) was centrifuged to collect the microbial cells. The collected cells were then suspended in 1000 μL of 100 mM borate buffer (pH 8.5 or pH 9.0) containing 10 mM EDTA, 100 mM L-alanine methyl ester and 200 mM glutamine, and reacted at 25° C. for 10 minutes. The concentrations of Ala-Gln synthesized with the wild strain in this reaction are shown in Table 5. For the dipeptide synthesized by various mutant strains, the ratio of the concentration of the dipeptide synthesized by the mutant strain with respect to that by the wild strain is shown in Table 5.

(17) Production of Peptide Using Microbial Cells <Phe-Met, Leu-Met>

800 μL of the cultured medium obtained in Example 4 (14) was centrifuged to collect the microbial cells. The collected cells were then suspended in 400 μL of 100 mM borate buffer (pH 8.5 or pH 9.0) containing 10 mM EDTA, 50 mM L-phenylalanine methyl ester hydrochloride or L-leucine methyl ester hydrochloride, and 100 mM L-methionine, and reacted at 25° C. for 20 minutes. The concentrations of Phe-Met and Leu-Met synthesized with the wild strain in this reaction are shown in Table 5. For the dipeptides synthesized by various mutant strains, the ratio of the concentration of the dipeptide synthesized by the mutant strain with respect to that by the wild strain is shown in Table 5.

TABLE 5
Table 5

| | | SYNTHESIZED DIPEPTIDE NAME | | | | | | | |
|---|---|---|---|---|---|---|---|---|---|
| | | AMP | | Ala-Gln | | Phe-Met | | Leu-Met | |
| REACTION pH | | 8.5 | 9.0 | 8.5 | 9.0 | 8.5 | 9.0 | 8.5 | 9.0 |
| PRODUCTION AMOUNT | | 3.7 | 0.9 | 3.0 | 1.8 | 2.4 | 1.9 | 8.5 | 8.5 |
| OF CONTROL ENZYME | | | | | | | | | |
| DIPEPTIDE [mM] | | | | | | | | | |
| RATIO OF THE | F207V | | 1.5 | | 0.1 | 2.3 | 2.3 | 2.9 | 2.5 |
| SYNTHESIZED | Q441E | 1.0 | 1.2 | 1.0 | 1.1 | 1.2 | 0.9 | 1.1 | 1.1 |
| DIPEPTIDE | F207V/Q441E | 0.7 | 2.1 | 0.8 | 0.4 | 2.7 | 2.9 | 3.5 | 3.0 |
| CONCENTRATION | K83A | | 1.6 | | 1.5 | 4.3 | 3.3 | 2.8 | 3.1 |
| IN VARIOUS | M208A | | | | | 4.2 | 2.1 | 1.2 | 1.0 |
| MUTANT STRAINS | F207H | | 4.0 | | 4.2 | | | | |
| TO THAT IN THE | K83A/F207V | 2.0 | 7.5 | 3.3 | 2.0 | 9.9 | 9.4 | 10.1 | 8.2 |
| WILD STRAIN* | K83A/Q441E | 2.6 | 3.8 | 2.9 | 3.1 | 2.6 | 2.1 | 1.7 | 1.9 |
| | K83A/F207V/Q441E | 2.0 | 6.9 | 2.8 | 1.8 | 4.8 | 5.0 | 5.5 | 5.2 |
| | L439V/F207V/Q441E | 2.5 | 12.7 | | | | | | |
| | A537G/F207V/Q441E | 2.3 | 13.0 | | | | | | |
| | A301V/F207V/Q441E | 2.8 | 16.0 | | | | | | |
| | G226S/F207V/Q441E | 2.3 | 12.6 | | | | | | |
| | V257I/F207V/Q441E | 2.3 | 16.5 | | | | | | |
| | D619E/F207V/Q441E | 2.4 | 13.2 | | | | | | |
| | Y339H/F207V/Q441E | 2.4 | 12.4 | | | | | | |
| | N607K/F207V/Q441E | 2.4 | 12.2 | | | | | | |
| | A324V/F207V/Q441E | 2.9 | 14.7 | | | | | | |
| | Q229H/F207V/Q441E | 3.5 | 21.9 | | | | | | |
| | W327G/F207V/Q441E | 2.1 | 10.8 | | | | | | |

*THIS SHOWS RATIO OF THE SYNTHESIZED DIPEPTIDE CONCENTRATION IN VARIOUS MUTANT STRAINS WHEN THE SYNTHESIZED DIPEPTIDE CONCENTRATION IN THE WILD STRAIN IS “1”

### Example 5

### Random Screening 2

(18) Preparation of pSTV_Sm_Aet Random Library

In order to construct mutant Aet, pHSG_Sm_Aet plasmid was used as the template for random mutagenesis using error prone PCR. The mutation was introduced using “GeneMorph PCR Mutagenesis Kit” supplied from Stratagene (USA) in accordance with the protocol of the manufacturer.

The PCR was performed using the oligonucleotides shown in SEQ ID NOS:3 and 4. That is, 100 ng of ds DNA (pHSG_Sm_Aet plasmid) as the template, 1.25 pmol each of the primers 1 and 2 and 2.5 units of Murazyme DNA polymerase were added to 50 μL of Mutazyme reaction buffer containing 200 M each of DATP, dCTP, dGTP and dTTP. The mixture was heated at 95° C. for 30 seconds and then subjected to the PCR using 25 cycles at 95° C. for 30 seconds, 52° C. for 30 seconds and 72° C. for 2 minutes.

The PCR product was treated with EcoRI/PstI, and the resulting DNA fragment was ligated to pSTV28 (suppled from Takara Shuzo Co., Ltd.) that had been treated with EcoRI/PstI. Escherichia coli JM109 was transformed with this solution containing the ligated product. This transformed strain was plated on M9 agar medium (200 mL/L of 5*M9, 1 mL/L of 0.1 M CaCl₂, 1 mL/L of 1 M MgSO₄, 10 mL/L of 50% glucose, 10 g/L of casamino acid, 15 g/L of agar) containing 50 pg/mL of chloramphenicol and 0.1 mM IPTG to make a library in which random mutation was introduced. At that time, for the sake of simplicity of the subsequent screening, the transformants were applied so that about 100 colonies per plate would be grown. The above “5*M9” is a solution containing 64 g/L of Na₂HPO₄.7H₂O, 15 g/L of KH₂PO₄, 2.5 g/L of NaCl and 5 g/L of NH₄Cl.

(19) Primary Screening From pSTV Based Random Library

In order to efficiently select the strain whose activity had been enhanced from the resulting transformants (library from mutant enzyme-expressing strain), Phe-pNA hydrolytic activity of each transformant was examined. A reaction solution (10 mM Phe-pNA, 10 mM OPT, 20 mM Tris-HCl (pH 8.2), 0.8% agar)(5 mL) was overlaid on the plate for transformant growth made in Example 5 (18), and color development by pNA produced by hydrolysis of Phe-pNA was examined (microbial cells are colored in yellow by liberation of pNA). The strongly colored colony was selected as the strain whose activity had been enhanced.

(20) Obtaining Microbial Cells

The selected strains were cultured on the LB agar medium at 30° C. for 24 hours. One platinum loopful of microbial cells of each strain was inoculated to 3 mL of the LB medium (agar was omitted from the above medium) containing 0.1 mM IPTG and 50 mg/L of chloramphenicol, and the main cultivation was performed at 25° C. at 150 reciprocatings/minute for 20 hours.

(21) Secondary Screening

Microbial cells were collected from 400 μL of the cultured broth obtained in Example 5 (20). The collected cells were suspended in 400 μL of 100 mM borate buffer (pH 9.0) containing 10 mM EDTA, 50 mM Phe-OMe and 100 mM Met, and reacted at 25° C. for 30 minutes. The amount of synthesized Phe-Met was measured, and the strains whose initial rate of the reaction was fast were selected. For the selected strains whose activity had been enhanced, the mutation point was analyzed, and the mutation points 11 and 12 were specified.

(22) Production of Peptide Using Microbial Cells <Phe-Met, Leu-Met>

800 μL of the cultured medium obtained in Example 5 (20) was centrifuged to collect the microbial cells. The collected cells were then suspended in 400 μL of 100 mM borate buffer (pH 9.0) containing 10 mM EDTA, 25 mM L-phenylalanine methyl ester hydrochloride or L-leucine methyl ester hydrochloride, and 50 mM L-methionine, and reacted at 25° C. for 20 minutes. The concentrations of Phe-Met and Leu-Met synthesized with the wild strain in this reaction are shown in Table 6. For the dipeptide synthesized by various mutant strains, the ratio of the concentration of the dipeptide synthesized by the mutant strain with respect to that by the wild strain is shown in Table 6.

TABLE 6
Table 6

| SYNTHESIZED DIPEPTIDE NAME | | Phe-Met | Leu-Met |
|---|---|---|---|
| PRODUCTION AMOUNT OF | | 1.35 mM | 4.86 mM |
| CONTROL ENZYME DIPEPTIDE | | | |
| RATIO OF THE | F207V | 1.6 | 1.6 |
| SYNTHESIZED | E551K | 2.2 | 1.4 |
| DIPEPTIDE | K83A/Q441E | 1.4 | 1.4 |
| CONCENTRATION IN | M208A/E551K | 5.3 | 2.4 |
| VARIOUS MUTANT | | | |
| STRAINS TO THAT IN | | | |
| THE WILD STRAIN* | | | |

*THIS SHOWS RATIO OF THE SYNTHESIZED DIPEPTIDE CONCENTRATION IN VARIOUS MUTANT STRAINS WHEN THE SYNTHESIZED DIPEPTIDE CONCENTRATION IN THE WILD STRAIN IS “1”

### Example 6

### High Expression of Peptide-Synthesizing Enzyme Gene in pSF_Sm_Aet

(23) Construction of Plasmid With High Expression

An expression plasmid was constructed by ligating the mature peptide-synthesizing enzyme gene derived from Sphingobacterium to downstream of a modified promoter and a signal sequence of acid phosphatase derived from Enterobacter aerogenes by PCR.

The peptide-synthesizing enzyme gene was amplified by PCR using 50 μL of a reaction solution containing 0.4 mM pTrpT_Sm_Aet (Example 1) as a template, 0.4 mM each of Esp-S1 (5′-CCG TAA GGA GGA ATG TAG ATG AAA AAT ACA ATT TCG TGC C; SEQ ID NO:121) and S-AS1 (5′-GGC TGC AGT TTG CGG GAT GGA AGG CCG GC; SEQ ID NO:122) oligonucleotides as the primers, KOD plus buffer (suppled from Toyobo Co., Ltd.), 0.2 mM each of DATP, dCTP, dGTP and dTTP, 1 mM magnesium sulfate and 1 unit of KOD plus polymerase (suppled from Toyobo Co., Ltd.), by heating at 94° C. for 30 seconds followed by 25 cycles at 94° C. for 15 seconds, 55° C. for 30 seconds and 68° C. for two minutes and 30 seconds. The promoter and signal sequences of acid phosphatase were amplified by PCR using pEAP130 plasmid (see the following Reference Example 1, related patent application: JP 2004-83481) as the template, and E-S1 (5′-CCT CTA GAA TTT TTT CAA TGT GAT TT; SEQ ID NO:123), and Esp-AS1 (5′-GCA GGA AAT TGT ATT TTT CAT CTA CAT TCC TCC TTA CGG TGT TAT; SEQ ID NO:124) oligonucleotides as the primers under the same condition as the above. The reaction solutions were subjected to agarose electrophoresis, and the amplified DNA fragments were recovered using Microspin column (supplied from Amersham Pharmacia Biotech).

Then, a chimeric enzyme gene was constructed by PCR using the amplified DNA fragment mixture as the template, E-S1 and S-AS1 oligonucleotides as the primer, and the reaction solution having the same composition as the above, for 25 cycles of 94° C. for 15 seconds, 55° C. for 30 seconds and 68° C. for two minutes and 30 seconds. The amplified DNA fragment was recovered using Microspin column (supplied from Amersham Pharmacia Biotech), and digested with XbaI and PstI. This was ligated to XbaI-PstI site of pCU18 plasmid. The nucleotide sequence was determined by a dye terminator method using a DNA sequencing kit, Dye Terminator Cycle Sequencing Ready Reaction (supplied from Perkin Elmer) and 310 Genetic Analyzer (ABI) to confirm that the objective mutation had been introduced, and then this plasmid was designated as pSF_Sm_Aet plasmid.

(24) Construction of Strain in Which pSF-Sm_Aet Rational Mutation has Been Introduced

To construct the mutant Aet, pSF_Sm_Aet was used as the template of site-directed mutagenesis using the PCR. The mutation was introduced using QuikChange Site-Directed Mutagenesis Kit supplied from Stratagene (USA) and the primers corresponding to each mutant enzyme (SEQ ID NOS:45 to 78) in accordance with the protocol of the manufacturer. Escherichia coli JM109 strain was transformed with PCR products, and strains having objective plasmids were selected with ampicillin resistance as the indicator. Escherichia coli JM109 strain having pSF_Sm_Aet is also represented as pSF_Sm_Aet/JM109 strain.

(25) Obtaining Microbial Cells

Each mutant strain obtained in Example 6 (24) was precultured in the LB agar medium at 25° C. for 16 hours. One platinum loopful of each strain expressing the enzyme was inoculated to 2 mL of terrific medium (12 g/L of tryptone, 24 g/L of yeast extract, 2.3 g/L of potassium dihydrogen phosphate, 12.5 g/L of dipotassium hydrogen phosphate, 4 g/L glycerol, 100 mg/L of ampicillin) in a general test tube, and the main cultivation was performed at 25° C. at 150 reciprocatings/minute for 18 hours.

(26) Production of Peptide Using Microbial Cells <Ala-Gln>

The cultured broth (5 μL) obtained in (25) was added to 500 μL of borate buffer (pH 8.5 or pH 9.0) containing 50 mM L-alanine methyl ester hydrochloride (A-OMe HCl), 100 mM L-glutamine and 10 mM EDTA, and reacted at 25° C. for 10 minutes. The concentrations of Ala-Gln synthesized with the wild strain in this reaction are shown in Table 7. For the dipeptide synthesized by various mutant strains, the ratio of the concentration of the dipeptide synthesized by the mutant strain with respect to that by the wild strain is shown in Table 7.

(27) Production of Peptide Using Microbial Cells <AMP>

The cultured broth (25 μL) obtained in the above was suspended in 500 μL of 100 mM borate buffer (pH 8.5 or pH 9.0) containing 10 mM EDTA, 50 mM dimethyl aspartate and 75 mM phenylalanine, and reacted at 20° C. or 25° C. for 15 minutes. The concentrations of AMP synthesized with the wild strain in this reaction are shown in Table 7. For the dipeptide synthesized by various mutant strains, the ratio of the concentration of the dipeptide synthesized by the mutant strain with respect to that by the wild strain is shown in Table 7.

(28) Production of Peptide Using Microbial Cells <Phe-Met, Leu-Met>

The cultured broth (25 μL) obtained in the above was suspended in 500 μL of 100 mM borate buffer (pH 8.5 or pH 9.0) containing 10 mM EDTA, 25 mM L-phenylalanine methyl ester hydrochloride or L-leucine methyl ester hydrochloride, and 50 mM L-methionine, and reacted at 25° C. for 15 minutes. The concentrations of Phe-Met and Leu-Met synthesized with the wild strain in this reaction are shown in Table 7. For the dipeptides synthesized by various mutant strains, the ratio of the concentration of the dipeptide synthesized by the mutant strain with respect to that by the wild strain is shown in Table 7.

TABLE 7
Table 7

| | | SYNTHESIZED DIPEPTIDE NAME | | | | | | | |
|---|---|---|---|---|---|---|---|---|---|
| | | AMP | | Ala-Gln | | Phe-Met | | Leu-Met | |
| REACTION pH | | 8.5 | 9.0 | 8.5 | 9.0 | 8.5 | 9.0 | 8.5 | 9.0 |
| PRODUCTION AMOUNT | | 9.5 | 3.7 | 18.9 | 17.1 | 1.5 | 1.9 | 9.4 | 10.1 |
| OF CONTROL ENZYME | | | | | | | | | |
| DIPEPTIDE [mM] | | | | | | | | | |
| RATIO OF THE | F207V/Q441E | 0.4 | 1.6 | 0.6 | 0.3 | 1.1 | 1.4 | 1.7 | 1.7 |
| SYNTHESIZED | K83A | 0.9 | 1.0 | 1.2 | 1.2 | 1.0 | 1.0 | 1.0 | 1.0 |
| DIPEPTIDE | A301V | 0.9 | 1.4 | 0.9 | 0.8 | 0.9 | 0.9 | 0.9 | 1.0 |
| CONCENTRATION | V257I | 1.0 | 2.0 | 1.0 | 1.0 | 1.0 | 1.0 | 1.0 | 1.1 |
| IN VARIOUS | A537G | 1.0 | 1.6 | 1.1 | 1.2 | 1.0 | 1.1 | 1.0 | 1.1 |
| MUTANT STRAINS | A324V | 1.0 | 1.4 | 1.3 | 1.1 | 1.1 | 1.1 | 1.0 | 1.0 |
| TO THAT IN THE | D313E | 1.0 | 1.2 | 1.2 | 1.2 | 1.1 | 1.0 | 1.1 | 1.0 |
| WILD STRAIN* | Q229H | 1.1 | 1.4 | 1.1 | 1.2 | 1.1 | 1.1 | 1.0 | 1.0 |
| | M208A | 0.5 | 0.3 | 0.7 | 0.2 | 4.5 | 2.6 | 1.1 | 0.9 |
| | E551K | 1.0 | 1.3 | 1.1 | 1.2 | 1.0 | 1.1 | 1.0 | 1.1 |
| | K83A/F207V | 0.5 | 1.5 | 0.6 | 0.3 | 1.1 | 1.3 | 1.7 | 1.7 |
| | E551K/F207V | 0.6 | 1.8 | 0.6 | 0.3 | 1.2 | 1.7 | 1.8 | 1.8 |
| | K83A/Q441E | 1.1 | 1.4 | 1.2 | 1.2 | 1.1 | 1.1 | 1.1 | 1.2 |
| | M208A/E551K | 0.7 | 0.4 | 0.8 | 0.2 | 5.2 | 3.9 | 1.3 | 1.2 |
| | V257I/Q441E | 1.1 | 2.1 | 1.1 | 1.2 | 0.9 | 1.2 | 1.1 | 1.1 |
| | K83A/F207V/Q441E | 0.6 | 1.8 | 0.8 | 0.4 | 1.3 | 1.5 | 1.8 | 1.9 |
| | L439V/F207V/Q441E | 0.6 | 1.6 | 0.7 | 0.3 | 1.3 | 1.4 | 1.8 | 1.7 |
| | A301V/F207V/Q441E | 0.6 | 1.8 | 0.5 | 0.4 | 1.2 | 1.4 | 1.8 | 1.9 |
| | G226S/F207V/Q441E | 0.6 | 1.8 | 0.7 | 0.4 | 1.1 | 1.5 | 1.8 | 1.8 |
| | V257I/F207V/Q441E | 0.5 | 1.8 | 0.6 | 0.5 | 1.0 | 1.3 | 1.8 | 1.9 |

*THIS SHOWS RATIO OF THE SYNTHESIZED DIPEPTIDE CONCENTRATION IN VARIOUS MUTANT STRAINS WHEN THE SYNTHESIZED DIPEPTIDE CONCENTRATION IN THE WILD STRAIN IS “1”

### Example 7

### Construction of Strain Having High Activity by Combination of Mutations

(29) Construction of Random Screening Mutation-Combining Strain

To construct strains where various mutations were combined, pSF_Sm_Aet was used as the template for site-directed mutagenesis using the PCR.

The mutation was introduced using “QuikChange Multi” supplied from Stratagene (USA) in accordance with the protocol of the manufacturer and using the primers (99 to 120) corresponding to each mutant enzyme. The 5′ terminus of the primers were phosphorylated before use with T4 polynucleotide kinase supplied from Takara Shuzo Co., Ltd. The primer was phosphorylated by adding 100 μmol DNA (primer) and 10 units of T4 polynucleotide kinase to 20 μL of 50 mM tris hydrochloric acid buffer (pH 8.0) containing 0.5 mM ATP, 10 mM magnesium chloride and 5 mM DTT and warming at 37° C. for 30 minutes followed by heating at 70° C. for 5 minutes.

The PCR was performed by adding 50 ng of ds DNA (pSF_Sm_Aet plasmid) as the template, 50 or 100 ng each of the 5′-phosphorylated mutagenic oligonucleotides (100 ng when the number of sort of primers in the combination is up to 3 types, and 50 ng when the number of sort of the primers in the combination is 4 types or more), 0.375 μL of Quik solution and 1.25 units of QuikChange Multi enzyme blend to 12.5 μL of QuckChange Multi reaction buffer containing 0.5 μL of dNTP mix, which was then subjected to the reaction of 30 cycles at 95° C. for one minute, 53.5° C. for one minute and 65° C. for 10 minutes.

Escherichia coli JM109 strain was transformed with 2 μL of the reaction solution obtained by adding 5 unites of DpnI to the PCR product (total amount: 12.5 μL) and treating at 37° C. for one hour. Transformed microbial cells were plated on the LB medium containing 100 μg/mL of ampicillin to obtain a library of randomly combined strains as ampicillin resistant strains.

(30) Screening From Library Having Combined Mutations

Escherichia coli JM109 strain transformed with the plasmid (pTrpT_Sm_AetM) containing each mutant Aet gene and Escherichia coli JM109 strain transformed with the plasmid containing the wild type Aet were inoculated to 150 μL (dispensed in wells of 96-well plate) of the medium containing 100 μg/mL of ampicillin, and cultured at 25° C. for 16 hours with shaking. The cultivation was performed with shaking at 1000 rotations/minute using a bio-shaker (M/BR-1212FP) supplied from TITEC. Using the resulting cultured medium, the selection was performed by screening.

(31) Primary Screening

A reaction solution (200 μL) (pH 8.2) containing 10 mM phenol, 6 mM AP, 5 mM Asp (OMe)₂, 7.5 mM Phe, 3.6 U/mL of peroxidase, 0.16 U/mL of alcohol oxidase, 10 mM EDTA and 100 mM borate was added to 5 μL of resulting microbial medium, which was then reacted at 25° C. for about 20 minutes. After the reaction, the absorbance at 500 nm was measured, and the amount of released methanol was calculated. Those showing the large amount of released methanol were selected as those having the enzyme with high AMP-synthesizing activity.

(32) Secondary Screening

After the primary screening described above, the selected strains were cultured by the method described in Example 6 (25). 10 μL or 50 μL of each cultured broth was suspended in 1 mL of 100 mM borate buffer (pH 8.5) containing 10 mM EDTA, 50 mM Asp(OMe)₂ and 75 mM Phe, and reacted at 20° C. or 25° C. for 10 minutes. The amount of synthesized AMP was measured and strains that exerted a large synthesis amount were selected. The combination of the mutation points was determined in the selected strains by sequencing. The obtained strains and the combinations of the primers used for obtaining the strains are shown in Table 8.

TABLE 8
Table 8

| OBTAINED | MOTHER | |
|---|---|---|
| STRAIN | STRAIN | PRIMER USED |
| M7-35 (260) | pSF 2458 | 2458 K83A F, 2458 Q229H F, |
| | | 2458 V257I F, 2458 A301V F, |
| | | 2458 D313E F, 2458 A324V F, |
| | | 2458 L439V F, 2458 Q441E F, |
| | | 2458 A537G F, 2458 N607K F |
| M7-46 (261) | pSF 2458 | 2458 K83A F, 2458 Q229H F, |
| | | 2458 V257I F, 2458 A301V F, |
| | | 2458 D313E F, 2458 A324V F, |
| | | 2458 L439V F, 2458 Q441E F, |
| | | 2458 A537G F, 2458 N607K F |
| M7-54 (262) | pSF 2458 | 2458 K83A F, 2458 Q229H F, |
| | | 2458 V257I F, 2458 A301V F, |
| | | 2458 D313E F, 2458 A324V F, |
| | | 2458 L439V F, 2458 Q441E F, |
| | | 2458 A537G F, 2458 N607K F |
| M7-63 (263) | pSF 2458 | 2458 K83A F, 2458 Q229H F, |
| | | 2458 V257I F, 2458 A301V F, |
| | | 2458 D313E F, 2458 A324V F, |
| | | 2458 L439V F, 2458 Q441E F, |
| | | 2458 A537G F, 2458 N607K F |
| M7-95 (264) | pSF 2458 | 2458 K83A F, 2458 Q229H F, |
| | | 2458 V257I F, 2458 A301V F, |
| | | 2458 D313E F, 2458 A324V F, |
| | | 2458 L439V F, 2458 Q441E F, |
| | | 2458 A537G F, 2458 N607K F |
| M9-9 (265) | M7-35 | T72A F, A137S F, 2458 Q441E F |
| M9-10 (266) | M7-35 | T72A F, A137S F, 2458 Q441E F |
| M11-2 (267) | M7-63 | T72A F, A137S F, 2458 L439V F |
| M11-3 (268) | M7-63 | T72A F, A137S F, 2458 L439V F |
| M12-1 (269) | M7-95 | T72A F, A137S F, 2458 L439V F |
| M12-3 (270) | M7-95 | T72A F, A137S F, 2458 L439V F |
| M21-18 (271) | M9-9 | Q229X F |
| M21-22 (272) | M9-9 | Q229X F |
| M21-25 (273) | M9-9 | Q229X F |
| M22-25 (274) | M12-1 | Q229X F |
| M24-1 (275) | M9-9 | I228X F + Q229P F |
| M24-2 (276) | M9-9 | I228X F + Q229P F |
| M24-5 (277) | M9-9 | I228X F + Q229P F |
| M26-3 (278) | M9-9 | I230X F + Q229P F |
| M26-5 (279) | M9-9 | I230X F + Q229P F |
| M29-3 (280) | M12-1 | I228X F + Q229H F |
| M33-1 (281) | M12-1 | S256X F + V257I F |
| M35-4 (282) | M11-3 | A137X F, 2458 V257I F, |
| | | 2458 Q229P F |
| M37-5 (283) | M11-3 | 2458 V257I F, 2458 Q229P F, |
| | | A324X F |
| M39-4 (284) | M12-3 | 2458 Q229P F, A301X F |
| M41-2 (285) | M12-3 | 2458 Q229P F, A537X F |

(33) Production of Peptide Using Microbial Cells

The combination strains obtained in the above were evaluated. The cultured broth (25 μL) obtained in the above was suspended in 500 μL of 100 mM borate buffer (pH 8.5) containing 10 mM EDTA, 50 mM dimethyl aspartate and 75 mM phenylalanine, and reacted at 20° C. for 15 minutes. The concentration of AMP synthesized with the wild strain in this reaction is shown in Table 9. For the dipeptide synthesized by various mutant strains, the ratio of the specific activity of the dipeptide synthesized by the mutant strain with respect to the specific activity as to the wild strain being 1 is shown in Table 9.

TABLE 9
Table 9

| | |
|---|---|
| | 20° C. |
| SYNTHESIZED DIPEPTIDE NAME | AMP |

| REACTION pH | | 8.5 |
|---|---|---|
| CELL AMOUNT | | 5% |
| PRODUCTION AMOUNT OF | | |
| CONTROL ENZYME DIPEPTIDE [mM] | | 7.8 |
| RATIO OF THE SYNTHESIZED | M7-35 | 4.8 |
| DIPEPTIDE CONCENTRATION IN | M7-46 | 3.7 |
| VARIOUS MUTANT STRAINS TO THAT | M7-54 | 1.9 |
| IN THE WILD STRAIN* | M7-63 | 5.3 |
| | M7-95 | 4.0 |
| | M9-9 | 6.1 |
| | M9-10 | 6.3 |
| | M11-2 | 6.0 |
| | M11-3 | 6.0 |
| | M12-1 | 6.4 |
| | M12-3 | 5.4 |
| | M21-18 | 5.7 |
| | M21-22 | 5.3 |
| | M21-25 | 3.7 |
| | M22-25 | 4.7 |
| | M24-1 | 6.7 |
| | M24-2 | 6.3 |
| | M24-5 | 7.2 |
| | M26-3 | 5.9 |
| | M26-5 | 7.6 |
| | M29-3 | 5.3 |
| | M33-1 | 5.5 |
| | M35-4 | 6.6 |
| | M37-5 | 7.2 |
| | M39-4 | 6.1 |
| | M41-2 | 5.8 |

### Example 8

### Study of Substrate Specificity

(34) Study of Substrate Specificity Using Mutant Enzyme

The production of peptides was examined in the cases of using various amino acid methyl ester for the carboxy component and L-methionine for the amine component. The cultured broth (25 μL) prepared by the method described in Example 6 (25) was added to 500 μL of borate buffer (pH 8.5) containing 25 mM L-amino acid methyl ester hydrochloride (X—OMe—HCl) shown in Table 10, 50 mM L-methionine and 10 mM EDTA. The mixture was then reacted at 25° C. for 15 minutes or 3 hours. The amounts of various peptides synthesized with the wild strain in this reaction are shown in Tables 10-1 and 10-2. The amount of the produced peptide with a mark “+” was not able to quantify because the standard samples were not available, and the amounts are thus shown in terms of estimated reference value of the peak, tentatively determining an area value of 8000 in HPLC being 1 mg/L. For the dipeptides synthesized by various mutant strains, the ratio of the concentration of the dipeptide synthesized by the mutant strain with respect to that by the wild strain is shown in Tables 10-1 and 10-2.

TABLE 10-1
Table 10-1

| | | SYNTHESIZED DIPEPTIDE NAME | | | | | | | |
|---|---|---|---|---|---|---|---|---|---|
| | | Ala-Met | | Ile-Met | | Leu-Met | | Met—Met | |
| | | REACTION TIME | | | | | | | |
| | | 15 | 3 | 15 | 3 | 15 | 3 | 15 | 3 |
| | | MIN | HRS | MIN | HRS | MIN | HRS | MIN | HRS |
| PRODUCTION AMOUNT OF | | 19.4 | 12.8 | 2.6 | 6.5 | 5.4 | 9.7 | 4.9 | 6.7 |
| CONTROL ENZYME | | | | | | | | | |
| DIPEPTIDE [mM] | | | | | | | | | |
| RATIO OF THE | F207V | 0.5 | 1.4 | 0.7 | 0.6 | 1.7 | 1.2 | 0.9 | 1.6 |
| SYNTHESIZED | Q441E | 0.9 | 0.9 | 1.0 | 1.6 | 1.1 | 0.9 | 1.2 | 1.3 |
| DIPEPTIDE | K83A | 0.9 | 1.0 | 1.3 | 1.3 | 1.2 | 0.8 | 1.2 | 1.1 |
| CONCENTRATION | A301V | 0.9 | 1.0 | 1.1 | 1.7 | 1.1 | 0.9 | 1.1 | 1.3 |
| IN VARIOUS | V257I | 1.0 | 0.8 | 1.1 | 2.4 | 1.2 | 0.6 | 1.1 | 1.7 |
| MUTANT STRAINS | A537G | 1.0 | 0.8 | 1.1 | 2.1 | 1.2 | 0.7 | 1.1 | 1.8 |
| TO THAT IN THE | A324V | 1.0 | 1.0 | 1.2 | 1.4 | 1.2 | 0.7 | 1.2 | 1.2 |
| WILD STRAIN* | N607K | 1.0 | 1.0 | 1.0 | 1.1 | 1.2 | 0.8 | 1.0 | 0.9 |
| | D313E | 1.0 | 1.0 | 1.1 | 1.5 | 1.3 | 0.7 | 1.0 | 1.1 |
| | Q229H | 1.0 | 1.0 | 0.9 | 1.4 | 1.2 | 0.7 | 0.9 | 1.3 |
| | M208A | 0.8 | 1.0 | 0.9 | 0.3 | 1.2 | 0.8 | 0.8 | 0.6 |
| | E551K | 1.0 | 1.2 | 1.2 | 1.5 | 1.1 | 0.9 | 1.0 | 1.2 |
| | F207V/Q441E | 0.6 | 1.4 | 0.9 | 0.8 | 1.8 | 1.3 | 1.1 | 1.7 |
| | K83A/F207V | | | | | 1.6 | 1.4 | | |
| | E551K/F207V | | | | | 1.6 | 1.2 | | |
| | K83A/Q441E | | | | | 1.0 | 1.1 | | |
| | M208A/E551K | | | | | 1.2 | 1.0 | | |
| | V257I/Q441E | | | | | 1.0 | 0.7 | | |
| | K83A/F207V/Q441E | | | | | 1.7 | 1.4 | | |
| | L439V/F207V/Q441E | | | | | 1.9 | 0.8 | | |
| | A301V/F207V/Q441E | | | | | 0.0 | 0.1 | | |
| | G226S/F207V/Q441E | | | | | 1.7 | 1.4 | | |
| | V257I/F207V/Q441E | | | | | 1.4 | 1.3 | | |
| | V257I/A537G | 1.0 | 0.9 | 0.0 | 0.0 | | | | |
| | M7-35 | 1.3 | 0.7 | 1.9 | 1.4 | | | | |
| | M7-46 | 1.2 | 0.8 | 1.3 | 1.4 | | | | |
| | M7-54 | 1.2 | 0.7 | 1.3 | 1.4 | | | | |
| | M7-63 | 1.3 | 0.6 | 2.1 | 1.4 | | | | |
| | M7-95 | 1.3 | 0.6 | 1.6 | 1.5 | | | | |
| | M9-9 | 1.3 | 0.6 | 3.3 | 1.4 | | | | |
| | M9-10 | 1.3 | 0.7 | 3.2 | 1.3 | | | | |
| | M11-2 | 1.3 | 0.6 | 3.1 | 1.3 | | | | |
| | M11-3 | 1.2 | 0.5 | 3.5 | 1.2 | | | | |
| | M12-1 | 1.3 | 0.5 | 3.0 | 1.3 | | | | |
| | M12-3 | 1.3 | 0.7 | 2.4 | 1.4 | | | | |
| | | SYNTHESIZED DIPEPTIDE NAME | | | | | | | |
| | | Phe-Met | | Pro-Met | | Trp-Met | | Val-Met | |
| | | REACTION TIME | | | | | | | |
| | | 15 | 3 | 15 | 3 | 15 | 3 | 15 | 3 |
| | | MIN | HRS | MIN | HRS | MIN | HRS | MIN | HRS |
| PRODUCTION AMOUNT OF | | 1.3 | 6.5 | 0.6 | 0.6 | 0.2 | 0.4 | 2.5 | 12.6 |
| CONTROL ENZYME | | | | | | | | | |
| DIPEPTIDE [mM] | | | | | | | | | |
| RATIO OF THE | F207V | 0.9 | 1.0 | 0.5 | 0.4 | 0.0 | 0.3 | 3.2 | 1.8 |
| SYNTHESIZED | Q441E | 1.0 | 0.9 | 0.9 | 1.3 | 1.2 | 1.4 | 1.0 | 1.2 |
| DIPEPTIDE | K83A | 1.1 | 0.9 | 0.9 | 1.1 | 1.0 | 1.1 | 1.3 | 1.1 |
| CONCENTRATION | A301V | 1.0 | 1.2 | 0.8 | 1.1 | 1.2 | 1.6 | 0.8 | 1.1 |
| IN VARIOUS | V257I | 1.2 | 1.3 | 0.9 | 1.7 | 1.5 | 3.0 | 1.0 | 1.1 |
| MUTANT STRAINS | A537G | 0.0 | 1.3 | 1.0 | 1.5 | 1.5 | 2.4 | 1.0 | 1.1 |
| TO THAT IN THE | A324V | 1.3 | 1.3 | 0.8 | 1.0 | 1.0 | 1.3 | 1.1 | 1.2 |
| WILD STRAIN* | N607K | 1.2 | 0.9 | 1.0 | 1.1 | 1.0 | 1.0 | 1.0 | 1.1 |
| | D313E | 1.2 | 1.3 | 0.9 | 1.2 | 1.1 | 1.3 | 1.1 | 1.1 |
| | Q229H | 1.3 | 1.3 | 0.9 | 1.3 | 1.2 | 1.6 | 1.1 | 1.2 |
| | M208A | 3.6 | 0.9 | 0.5 | 0.4 | 0.6 | 0.5 | 4.8 | 1.2 |
| | E551K | 1.0 | 1.3 | 0.9 | 1.0 | 1.2 | 1.6 | 1.2 | 1.2 |
| | F207V/Q441E | 1.0 | 1.1 | 0.5 | 0.4 | 0.0 | 0.6 | 3.6 | 1.7 |
| | K83A/F207V | 1.5 | 0.9 | | | | | 3.1 | 1.5 |
| | E551K/F207V | 1.7 | 1.1 | | | | | 2.7 | 1.5 |
| | K83A/Q441E | 1.3 | 0.9 | | | | | 0.9 | 1.0 |
| | M208A/E551K | 6.4 | 1.3 | | | | | 3.9 | 1.1 |
| | V257I/Q441E | 1.4 | 1.1 | | | | | 0.6 | 0.9 |
| | K83A/F207V/Q441E | 1.5 | 1.1 | | | | | 3.5 | 1.6 |
| | L439V/F207V/Q441E | 1.4 | 0.9 | | | | | 2.7 | 1.5 |
| | A301V/F207V/Q441E | 1.3 | 1.3 | | | | | 2.6 | 1.6 |
| | G226S/F207V/Q441E | 0.8 | 1.2 | | | | | 2.9 | 1.7 |
| | V257I/F207V/Q441E | 0.7 | 1.0 | | | | | 2.4 | 1.6 |
| | V257I/A537G | | | | | | | 0.0 | 0.0 |
| | M7-35 | | | | | | | 1.9 | 1.0 |
| | M7-46 | | | | | | | 1.2 | 1.1 |
| | M7-54 | | | | | | | 1.2 | 1.1 |
| | M7-63 | | | | | | | 2.2 | 0.9 |
| | M7-95 | | | | | | | 1.6 | 1.0 |
| | M9-9 | | | | | | | 3.1 | 0.7 |
| | M9-10 | | | | | | | 3.1 | 0.7 |
| | M11-2 | | | | | | | 3.0 | 0.8 |
| | M11-3 | | | | | | | 3.5 | 0.7 |
| | M12-1 | | | | | | | 3.0 | 0.7 |
| | M12-3 | | | | | | | 2.3 | 0.9 |

*THIS SHOWS RATIO OF THE SYNTHESIZED DIPEPTIDE CONCENTRATION IN VARIOUS MUTANT STRAINS WHEN THE SYNTHESIZED DIPEPTIDE CONCENTRATION IN THE WILD STRAIN IS “1”

TABLE 10-2
Table 10-2

| | | + | | + | | + | | | | | | | |
|---|---|---|---|---|---|---|---|---|---|---|---|---|---|
| | | SYNTHESIZED DIPEPTIDE NAME | | | | | | | | | | | |
| | | Asn-Met | | Cys-Met | | Gln-Met | | Gly-Met | | Ser-Met | | Thr-Met | |
| | | REACTION TIME | | | | | | | | | | | |
| | | 15 | 3 | 15 | 3 | 15 | 3 | 15 | 3 | 15 | 3 | 15 | 3 |
| | | MIN | HRS | MIN | HRS | MIN | HRS | MIN | HRS | MIN | HRS | MIN | HRS |
| PRODUCTION AMOUNT OF | | 1.4 | 2.2 | 8.6 | 10.9 | 2.8 | 5.1 | 8.2 | 13.8 | 0.7 | 1.2 | 7.3 | 11.9 |
| CONTROL ENZYME | | | | | | | | | | | | | |
| DIPEPTIDE [mM] | | | | | | | | | | | | | |
| RATIO OF THE | F207V | 0.0 | 0.1 | 0.5 | 0.7 | 0.9 | 1.0 | 0.0 | 0.1 | 0.0 | 0.0 | 0.0 | 0.0 |
| SYNTHESIZED | Q441E | 1.5 | 1.2 | 1.4 | 1.2 | 1.0 | 1.1 | 1.0 | 1.1 | 0.7 | 1.4 | 1.0 | 1.2 |
| DIPEPTIDE | K83A | 1.3 | 1.0 | 1.2 | 1.1 | 0.9 | 1.0 | 1.1 | 1.0 | 1.2 | 1.1 | 1.1 | 1.1 |
| CONCENTRATION | A301V | 1.1 | 1.2 | 1.1 | 1.1 | 1.0 | 1.1 | 1.0 | 1.3 | 1.0 | 1.7 | 1.1 | 0.0 |
| IN VARIOUS | V257I | 1.4 | 1.9 | 1.2 | 1.1 | 0.9 | 1.1 | 1.3 | 1.5 | 1.4 | 3.4 | 1.3 | 1.6 |
| MUTANT STRAINS | A537G | 1.5 | 1.7 | 1.3 | 1.1 | 1.0 | 1.2 | 1.3 | 1.5 | 1.4 | 2.6 | 1.2 | 1.7 |
| TO THAT IN THE | A324V | 1.5 | 1.1 | 1.4 | 1.1 | 1.2 | 1.2 | 1.3 | 1.2 | 1.1 | 1.4 | 1.2 | 1.3 |
| WILD STRAIN* | N607K | 1.1 | 1.0 | 1.1 | 1.1 | 0.8 | 1.0 | 1.1 | 1.0 | 1.1 | 1.2 | 1.0 | 1.0 |
| | D313E | 1.2 | 1.2 | 1.1 | 1.1 | 1.0 | 1.0 | 1.2 | 1.2 | 1.3 | 1.6 | 1.2 | 1.3 |
| | Q229H | 1.2 | 1.4 | 1.1 | 1.2 | 0.9 | 1.1 | 1.3 | 1.3 | 1.2 | 1.8 | 1.1 | 1.5 |
| | M208A | 0.1 | 0.1 | 0.4 | 0.3 | 0.7 | 0.6 | 0.0 | 0.0 | 0.0 | 0.0 | 0.0 | 0.0 |
| | E551K | 1.0 | 1.2 | 1.1 | 1.1 | 1.0 | 1.1 | 1.0 | 1.1 | 1.0 | 1.2 | 1.1 | 1.3 |
| | F207V/Q441E | 0.0 | 0.1 | 0.5 | 1.1 | 0.9 | 1.1 | 0.0 | 0.1 | 0.0 | 0.0 | 0.0 | 0.0 |
| | K83A/F207V | | | | | | | | | | | | |
| | E551K/F207V | | | | | | | | | | | | |
| | K83A/Q441E | | | | | | | | | | | | |
| | M208A/E551K | | | | | | | | | | | | |
| | V257I/Q441E | | | | | | | | | | | | |
| | K83A/F207V/Q441E | | | | | | | | | | | | |
| | L439V/F207V/Q441E | | | | | | | | | | | | |
| | A301V/F207V/Q441E | | | | | | | | | | | | |
| | G226S/F207V/Q441E | | | | | | | | | | | | |
| | V257I/F207V/Q441E | | | | | | | | | | | | |
| | V257I/A537G | | | 1.1 | 1.9 | | | 1.2 | 2.4 | | | | |
| | M7-35 | | | 2.2 | 2.1 | | | 2.8 | 2.5 | | | | |
| | M7-46 | | | 1.6 | 2.0 | | | 1.6 | 2.5 | | | | |
| | M7-54 | | | 2.0 | 1.9 | | | 1.6 | 2.6 | | | | |
| | M7-63 | | | 2.8 | 1.7 | | | 2.6 | 2.5 | | | | |
| | M7-95 | | | 2.5 | 1.7 | | | 2.1 | 2.6 | | | | |
| | M9-9 | | | 3.2 | 1.6 | | | 2.9 | 2.5 | | | | |
| | M9-10 | | | 2.3 | 2.0 | | | 1.7 | 2.5 | | | | |
| | M11-2 | | | 3.0 | 1.6 | | | 2.9 | 2.3 | | | | |
| | M11-3 | | | 3.1 | 1.5 | | | 2.9 | 2.3 | | | | |
| | M12-1 | | | 2.8 | 1.5 | | | 2.7 | 2.5 | | | | |
| | M12-3 | | | 2.6 | 1.7 | | | 1.9 | 2.4 | | | | |

| | |
|---|---|
| + | + |

| SYNTHESIZED DIPEPTIDE NAME | | | | |
|---|---|---|---|---|
| Tyr-Met | Asp-Met | Arg-Met | His-Met | Lys-Met |

REACTION TIME

| | | 15 | 3 | 15 | 3 | 15 | 3 | 15 | 3 | 15 | 3 |
|---|---|---|---|---|---|---|---|---|---|---|---|
| | | MIN | HRS | MIN | HRS | MIN | HRS | MIN | HRS | MIN | HRS |
| PRODUCTION AMOUNT OF | | 0.6 | 0.6 | 3.4 | 5.2 | 0.3 | 0.2 | 0.1 | 0.2 | 0.2 | 0.2 |
| CONTROL ENZYME | | | | | | | | | | | |
| DIPEPTIDE [mM] | | | | | | | | | | | |
| RATIO OF THE | F207V | 0.0 | 0.0 | 0.7 | 1.0 | 0.1 | 0.2 | 0.0 | 0.1 | 0.4 | 0.6 |
| SYNTHESIZED | Q441E | 1.8 | 1.9 | 1.1 | 1.3 | 1.2 | 0.8 | 1.5 | 1.2 | 0.8 | 2.2 |
| DIPEPTIDE | K83A | 1.6 | 1.7 | 1.1 | 1.1 | 1.0 | 1.3 | 1.5 | 1.1 | 0.9 | 1.7 |
| CONCENTRATION | A301V | 2.0 | 2.4 | 1.1 | 1.5 | 1.1 | 0.8 | 2.0 | 1.7 | 1.1 | 1.8 |
| IN VARIOUS | V257I | 3.3 | 5.6 | 1.2 | 1.7 | 2.1 | 4.7 | 3.1 | 4.6 | 0.0 | 8.5 |
| MUTANT STRAINS | A537G | 2.6 | 3.4 | 1.2 | 1.7 | 1.4 | 2.8 | 2.0 | 2.4 | 0.9 | 3.9 |
| TO THAT IN THE | A324V | 2.0 | 2.1 | 1.3 | 1.5 | 1.3 | 1.2 | 2.0 | 1.6 | 1.1 | 1.7 |
| WILD STRAIN* | N607K | 1.5 | 1.5 | 1.1 | 1.1 | 0.8 | 0.5 | 1.1 | 0.9 | 0.5 | 1.5 |
| | D313E | 1.7 | 2.0 | 1.2 | 1.4 | 0.8 | 1.3 | 1.0 | 0.8 | 1.1 | 2.0 |
| | Q229H | 1.8 | 1.9 | 1.2 | 1.5 | 1.4 | 1.8 | 1.4 | 1.2 | 1.7 | 2.3 |
| | M208A | 0.5 | 0.5 | 0.6 | 0.4 | 0.4 | 0.3 | 0.0 | 0.0 | 0.0 | 0.1 |
| | E551K | 1.5 | 1.6 | 1.1 | 1.3 | 1.0 | 0.9 | 1.5 | 1.2 | 1.1 | 1.6 |
| | F207V/Q441E | 0.0 | 0.0 | 0.7 | 1.1 | 0.0 | 0.1 | 0.1 | 0.2 | 0.3 | 0.3 |
| | K83A/F207V | | | | | | | | | | |
| | E551K/F207V | | | | | | | | | | |
| | K83A/Q441E | | | | | | | | | | |
| | M208A/E551K | | | | | | | | | | |
| | V257I/Q441E | | | | | | | | | | |
| | K83A/F207V/Q441E | | | | | | | | | | |
| | L439V/F207V/Q441E | | | | | | | | | | |
| | A301V/F207V/Q441E | | | | | | | | | | |
| | G226S/F207V/Q441E | | | | | | | | | | |
| | V257I/F207V/Q441E | | | | | | | | | | |
| | V257I/A537G | | | | | 2.7 | 6.3 | | | | |
| | M7-35 | | | | | 7.7 | 7.4 | | | | |
| | M7-46 | | | | | 7.0 | 13.6 | | | | |
| | M7-54 | | | | | 9.1 | 20.4 | | | | |
| | M7-63 | | | | | 15.0 | 21.8 | | | | |
| | M7-95 | | | | | 11.1 | 23.1 | | | | |
| | M9-9 | | | | | 16.6 | 23.3 | | | | |
| | M9-10 | | | | | 8.6 | 14.4 | | | | |
| | M11-2 | | | | | 19.2 | 24.1 | | | | |
| | M11-3 | | | | | 19.8 | 24.1 | | | | |
| | M12-1 | | | | | 18.8 | 22.8 | | | | |
| | M12-3 | | | | | 13.2 | 21.7 | | | | |

*THIS SHOWS RATIO OF THE SYNTHESIZED DIPEPTIDE CONCENTRATION IN VARIOUS MUTANT STRAINS WHEN THE SYNTHESIZED DIPEPTIDE CONCENTRATION IN THE WILD STRAIN IS “1”

### Example 9

### Random Screening

(35) Screening From pTrpT_Sm_Aet Random Library: B

The library produced in Example 3 (8) was cultured in the same way as in Example 3 (9), and two types of screenings were performed using the cultured medium.

(36) Primary Screening: A

A reaction solution (200 μL) (pH 8.2) containing 10 mM phenol, 6 mM AP, 5 mM Asp(OMe)₂, 5 mM Ala-OEt, 7.5 mM Phe, 3.6 U/mL of peroxidase, 0.16 U/mL of alcohol oxidase, 10 mM EDTA and 100 mM borate was added to 5 μL of the resulting microbial medium, which was then reacted at 25° C. for about 20 minutes. After the reaction, the absorbance at 500 nm was measured, and an amount of released methanol was calculated. Herein, those showing the large amount of released methanol were selected as those having the enzyme which tends to synthesize AMP more abundantly than Ala-Phe.

(37) Primary Screening: B

A reaction solution (200 μL) (pH 8.2) containing 10 mM phenol, 6 mM AP, 5 mM Asp(OMe) 2, 5mM A(M), 3.6 U/mL of peroxidase, 0.16 U/mL of alcohol oxidase, 10 mM EDTA and 100 mM borate was added to 5 μL of the resulting microbial medium, which was then reacted at 25° C. for about 20 minutes. After the reaction, the absorbance at 500 nm was measured, and an amount of released methanol was calculated. Herein, those showing the small amount of released methanol were selected as enzymes which has less tendency to produce AM (AM).

(38) Secondary Screening

The strains selected in Example 9 (36) and (37) were cultured in the same way as in Example 6 (25), and 50 μL of each cultured broth was suspended in 1 mL of 100 mM borate buffer (pH 8.5) containing 10 mM EDTA, 50 mM Asp(OMe)₂, 50 mM Ala-OMe and 75 mM Phe, and reacted 20° C. for 10 minutes. The amounts of synthesized AMP and Ala-Phe were measured, and the strains whose initial rate of the reaction was fast were selected. Likewise, 50 μL of each cultured broth was suspended in 1 mL of 100 mM borate buffer (pH 9.0) containing 10 mM EDTA, 50 mM Asp(OMe)₂, and 75 mM Phe, and reacted at 20° C. for 10 minutes. The yields of synthesized AMP were measured, and the strains exerting the high yield were selected. The mutation 21 was selected as the valid mutation point.

### Example 10

### Evaluation of Specified Mutation Point by Introducing it into pSF

(39) Introduction of Mutation into V184

The mutation point, V184A obtained in Example 9 was introduced into pSF_Sm_Aet, and also introduced into an existing construct, pSF_Sm_M35-4. V184X strains were also constructed by substituting V184 with other amino acids. The mutation was introduced in the same way as in (2) using pSF_Sm_Aet or pSF_Sm_M35-4 as the template and using the primers (SEQ ID NO:79 to 98) corresponding to each mutant enzyme. The resulting strains were cultured by the method described in Example 6 (25).

(40) Production of Peptide Using Microbial Cells <AMP>

The cultured broth (25 μL) prepared by the method described in Example 6 (24) was suspended in 500 μL of 100 mM borate buffer (pH 8.5 or pH 9.0) containing 10 mM EDTA, 50 mM dimethyl aspartate and 75 mM phenylalanine, and reacted at 20° C. for 10 minutes. The concentrations of AMP synthesized with the wild strain in this reaction are shown in Table 11. For the dipeptide synthesized by various mutant strains, the ratio of the concentration of the dipeptide synthesized by the mutant strain with respect to that by the wild strain is shown in Table 11.

TABLE 11
Table 11

| SYNTHESIZED DIPEPTIDE NAME | | AMP | AMP |
|---|---|---|---|
| pH | | 8.5 | 9 |
| PRODUCTION AMOUNT OF CONTROL | | 2.5 | 2.5 |
| ENZYME DIPEPTIDE [mM] | | | |
| RATIO OF THE | V184A | 6.1 | 2.9 |
| SYNTHESIZED | V184C | 1.6 | 1.0 |
| DIPEPTIDE | V184G | 0.8 | 0.1 |
| CONCENTRATION IN | V184I | 2.0 | 1.7 |
| VARIOUS MUTANT | V184L | 2.2 | 1.1 |
| STRAINS TO THAT | V184M | 3.7 | 1.1 |
| IN THE WILD | V184P | 1.6 | 0.9 |
| STRAIN* | V184S | 3.2 | 0.6 |
| | V184T | 3.2 | 0.3 |
| | M35-4 | | 5.7 |
| | M35-4/V184A | | 7.1 |
| | M35-4/V184G | | 1.7 |
| | M35-4/V184S | | 3.4 |
| | M35-4/V184T | | 6.2 |

*THIS SHOWS RATIO OF THE SYNTHESIZED DIPEPTIDE CONCENTRATION IN VARIOUS MUTANT STRAINS WHEN THAT SYNTHESIZED DIPEPTIDE CONCENTRATION IN THE WILD STRAIN IS “1”

(41) Production of Peptide Using Microbial Cells <AMP>

The cultured broth obtained by the method described in Example 6 (25) was suspended in 100 mM borate buffer (pH 8.5 or pH 9.0) containing 10 mM EDTA, 50 mM dimethyl aspartate and 75 mM phenylalanine, and reacted at 20° C. The yields of AMP synthesized with the wild strain and various mutant strains in this reaction are shown in Table 12.

TABLE 12
Table 12

| SYNTHESIZED DIPEPTIDE | | |
|---|---|---|
| NAME | AMP | AMP |
| pH | 8.5 | 9 |
| YIELD | 36.8 | 57.0% |
| V184A | 55.5 | 73.3 |
| V184C | 54.9 | |
| V184G | 64.3 | |
| V184I | 46.0 | |
| V184L | 44.5 | |
| V184M | 56.3 | |
| V184P | 54.6 | |
| V184S | 61.6 | |
| V184T | 60.3 | |
| M35-4 | | 57.5 |
| M35-4/V184A | | 68.8 |
| M35-4/V184G | | 77.2 |
| M35-4/V184N | | 77.3 |
| M35-4/V184S | | 70.8 |
| M35-4/V184T | | 67.7 |

### Example 11

### Change of Natures in Mutant Enzymes

(42) pH Stability of Enzymes

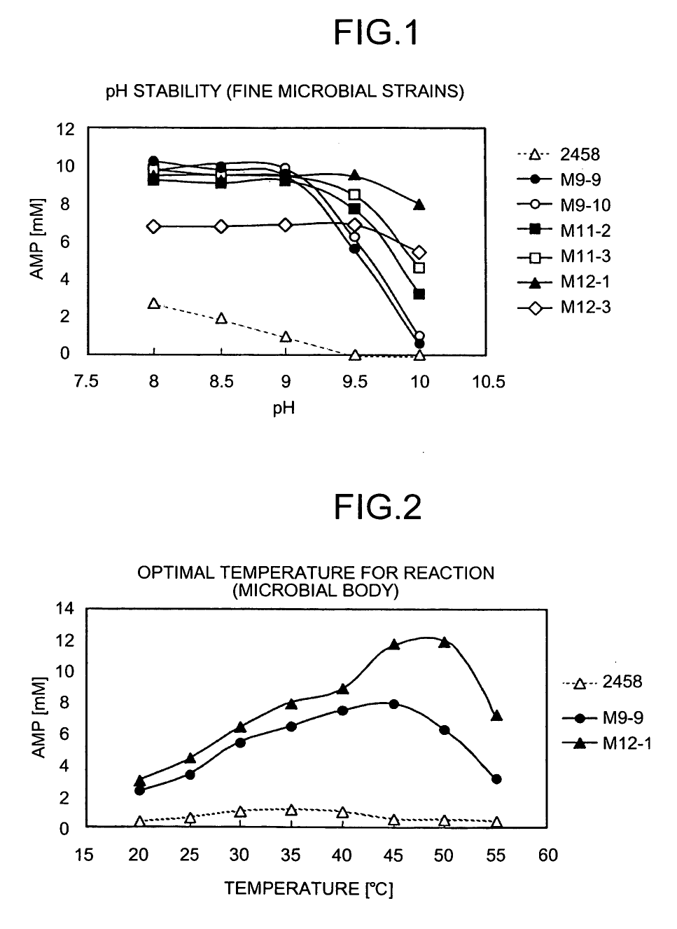

pH Stability was examined by incubating the enzyme at a certain pH for a certain period of time and subsequently synthesizing AMP from dimethyl L-aspartate hydrochloride and L-phenylalanine. The cultured broth (10 μL) prepared by the method described in Example 6 (25) was mixed with 190 μL of each of buffers at a variety of pH's (8.5, 9.0, 9.5) (as to M9-9 and M12-1, pH 8.0 was also tested), incubated for 30 minutes, and subsequently added to 400 μL of 450 mM borate buffer containing 75 mM dimethyl L-aspartate, 112.5 mM L-phenylalanine and 15 mM EDTA, which was then reacted at 20° C. for 20 minutes. The concentrations of synthesized AMP are shown in FIG. 1.

(43) Optimal Reaction Temperature of Enzymes

Effects of the reaction temperature on the reaction to synthesize AMP from dimethyl L-aspartate hydrochloride and L-phenylalanine were examined. The cultured broth (20 μL) prepared by the method described in Example 6 (25) was added to 980 μL of 100 mM borate buffer (pH 8.5) containing 50 mM dimethyl L-aspartate, 75 mM L-phenylalanine and 10 mM EDTA, and reacted at each temperature (20, 25, 30, 35, 40, 45, 50, 55, 60° C.) for 5 minutes. The concentrations of synthesized AMP are shown in FIG. 2. As a result, the optimal temperatures of the present enzymes were 35° C., 45° C. and 50° C. for 2458, M9-9 and M12-1, respectively.

(44) Temperature Stability of Enzymes

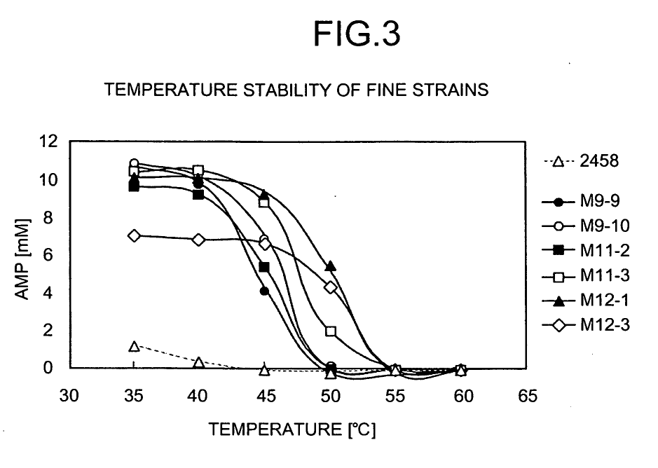

Temperature stability was examined by incubating the enzymes at a certain temperature for a certain period of time and subsequently synthesizing AMP from dimethyl L-aspartate hydrochloride and L-phenylalanine. The cultured broth (20 μL) that had been prepared by the method described in Example 6 (25) was incubated at each temperature (35, 40, 45, 50, 55, 60° C.) for 30 minutes, and was subsequently added to 980 μL of 100 mM borate buffer (pH 8.5) containing 50 mM dimethyl L-aspartate, 100 mM L-phenylalanine and 10 mM EDTA, which was then reacted at 20° C. for 5 minutes. The concentrations of AMP synthesized thereby are shown in FIG. 3.

<Analysis of Products>

In the aforementioned Examples, the products were quantified by the high performance liquid chromatography, details of which are as follows. Column: Inertsil ODS-3 (supplied from GL Sciences), eluants: i) aqueous solution of phosphoric acid containing 5.0 mM sodium 1-octanesulfonate (pH 2.1): methanol=100:15 to 50, ii) aqueous solution of phosphoric acid containing 5.0 mM sodium 1-octanesulfonate (pH 2.1): acetonitrile=100:15 to 30, flow rate: 1.0 mL/minute, and detection: 210 nm.

<Reference Example: Preparation of pEAP130 Plasmid—Modification of Promoter Sequence of Acid Phosphatase Gene Derived from Enterobacter aerogenes>

In accordance with the description of Journal of Bioscience and Bioengineering, 92(1):50-54, 2001 (or JP H10-201481 A publication), a DNA fragment of 1.6 kbp which contains an acid phosphatase gene region was cleaved out and isolated with restriction enzymes SalI and KpnI from a chromosomal DNA derived from Enterobacter aerogenes IFO 12010 strain. The fragment was ligated to pUC118 to construct a plasmid DNA which was designated as pEAP120. The nucleotide sequences encoding the promoter and the signal peptide of acid phosphatase were incorporated into the plasmid pEAP120. The strain to which IFO number was given has been deposited to Institute for Fermentation (17-85 Joso-honnmachi, Yodogawa-ku, Osaka, Japan), but, its operation has been transferred to NITE Biological Resource Center (NBRC), Department of Biotechnology (DOB), National Institute of Technology and Evaluation since Jun. 30, 2002, and the strain can be furnished from NBRC with reference to the above IFO number.

Subsequently, it was attempted to enhance the activity by partially modifying the promoter sequence present upstream of this gene. The site-directed mutation was introduced using QuikChange Site-Directed Mutagenesis Kit (supplied from Stratagene) to replace—10 region of the putative promoter sequence of the acid phosphatase gene from AAAAAT to TATAAT. Oligonucleotide primers for PCR, EM1 (5′-CTT ACA GAT GAC TAT AAT GTG ACT AAA AAC: SEQ ID NO:125) and EMR1 (5′-GTT TTT AGT CAC ATT ATA GTC ATC TGT AAG: SEQ ID NO:126) designed for introducing the mutation were synthesized. In accordance with the method of the instructions, the mutation was introduced using pEAP120 as the template. The nucleotide sequence was determined by the dye termination method using DNA Sequencing Kit Dye Terminator Cycle Sequencing Ready Reaction (supplied from Perkin Elmer) and using 310 Genetic analyzer (ABI) to confirm that the objective mutation had been introduced, and this plasmid was designated as pEAP130. The plasmid pEAP130 has the nucleotide sequences encoding the signal peptide and the modified promoter derived from the N terminal region of acid phosphatase.

<Abbreviation List>

Asp(OMe)₂ HCl: L-aspartic acid-α,β-dimethyl ester hydrochloride
Ala-OEt: L-alanine-ethyl ester
AMP: α-L-aspartyl-L-phenylalanine-β-ester
Ala-Gln: L-alanyl-L-glutamine
Ala-Phe: L-alanyl-L-phenylalanine
Phe-Met: L-phenylalanyl-L-methionine
Leu-Met: L-leucyl-L-methionine
Ile-Met: L-isoleucyl-L-methionine
Met-Met: L-methionyl-L-methionine
Pro-Met: L-prolyl-L-methionine
Trp-Met: L-tryptophanyl-L-methionine
Val-Met: L-valyl-L-methionine
Asn-Met: L-asparaginyl-L-methionine
Cys-Met: L-cysteinyl-L-methionine
Gln-Met: L-glutamyl-L-methionine
Gly-Met: L-glycyl-L-methionine
Ser-Met: L-seryl-L-methionine
Thr-Met: L-threonyl-L-methionine
Tyr-Met: L-tyrosinyl-L-methionine
Asp-Met: L-aspartyl-L-methionine
Arg-Met: L-arginyl-L-methionine
His-Met: L-histidyl-L-methionine
Lys-Met: L-lysyl-L-methionine
Ap: 4-aminoantipyrine
OPT: 1,10-phenanthoroline monohydrate
   [Sequence Listing Free Text]

Primer sequence list

TABLE 13-1

Table 13-1: PRIMER LIST
(No. IN THE LIST INDICATES SEQUENCE NUMBER)

| | | |
|---|---|---|
| No. | Name | Sequence |

| | | |
|---|---|---|
| 3 | 2458 EcoRI-S | CGCGAATTCATGAAAAATACAATTTCGTGC |
| 4 | 2458 PstI-AS | CGCCTGCAGCTAATCTTTGAGGACAGAAAATTC |
| 5 | 2458 NdeI F | GGGAATTCCATATGAAAAATACAATTTCGT |
| 6 | 2458 XbaI R | GCTCTAGACTAATCTTTGAGGACAGAAAA |
| 7 | 2458 Check F2 | TGCTCAATAGAACGCCCTA |
| 8 | 2458 Check F3 | CCGAGCTTGAAGGCAGTCT |
| 9 | 2458 Check F4 | ACGCGGAAGATGCTTATGG |
| 10 | 2458 Check F5 | AAGTTCAACGTACAGATT |
| 11 | 2458 Check R4 | GGTATCCGTACTTTCATCGA |

TABLE 13-2

Table 13-2: PRIMER LIST
(No. IN THE LIST INDICATES SEQUENCE NUMBER)

| | INTRODUCED | |
|---|---|---|
| No. | MUTATION | Sequence |
| 12 | S209D | GCA TTT ACA TTC ATG GAC ACC TTT GGT GTC CCT CG |
| 13 | Q441E | CAA GGT GGG TTA ATT GAA AAC CGA ACA CGG GAG |
| 14 | Q441K | CAA GGT GGG TTA ATT AAA AAC CGA ACA CGG GAG |
| 15 | N442K | GGT GGG TTA ATT CAA AAA CGA ACA CGG GAG TAT ATG |
| 16 | R445D | CAA AAC CGA ACA GAG GAG TAT ATG GTA GAT G |
| 17 | R445F | CAA AAC CGA ACA TTT GAG TAT ATG GTA GAT G |
| 18 | D203N | GTA TTG TTT CTT CAG AAT GCA TTT ACA TTC ATG |
| 19 | D203S | GTA TTG TTT CTT CAG TCT GCA TTT ACA TTC ATG |
| 20 | F207A | CAG GAT GCA TTT ACA GCC ATG TCA ACC TTT GGT G |
| 21 | F207S | CAG GAT GCA TTT ACA TCC ATG TCA ACC TTT GGT G |
| 22 | S209A | GCA TTT ACA TTC ATG GCA ACC TTT GGT GTC CCT C |
| 23 | Q441N | CAA GGT GGG TTA ATT AAC AAC CGA ACA CGG GAG |
| 24 | Q441D | CAA GGT GGG TTA ATT GAC AAG CGA ACA CGG GAG |
| 25 | K83A | CAG AAC GAA TAC AAA GCA AGT TTG GGA AAC |
| 26 | F207V | CAG GAT GCA TTT ACA GTC ATG TCA ACC TTT GGT G |
| 27 | F207G | CAG GAT GCA TTT ACA GGC ATG TCA ACC TTT GGT G |
| 28 | F207T | CAG GAT GCA TTT ACA ACC ATG TCA ACC TTT GGT G |
| 29 | M208A | GAT GCA TTT ACA TTC GCG TCA ACC TTT GGT GTC |
| 30 | 5209G | GCA TTT ACA TTC ATG GGA ACC TTT GGT GTC CC |
| 31 | F207I | CAG GAT GCA TTT ACA ATC ATG TCA ACC TTT GGT G |
| 32 | R117A | GATTTTGAAGATATAGCTCCGACCACGTACAGC |
| 33 | F207V/S209A | CAG GAT GCA TTT ACA GTC ATG GCA ACC TTT GGT G |
| 34 | L439V | CAA GGT GGG GTA ATT CAA AAC |
| 35 | A537G | CGA TAA AGG GCA GGC CTT G |
| 36 | A301V | GCG GAA GAT GTT TAT GGA AC |
| 37 | G226S | CAA TTT AAG AGC AAA ATT C |
| 38 | V257I | GGT GAC TCC ATA CAA TTT TG |
| 39 | D619E | TTT CTG TCC TCA AA G AAT AG |
| 40 | Y339H | GAA GGA AAC CAT TTA GGT G |
| 41 | N607K | CAC GAT GTG AAG AAT GCC AC |
| 42 | A324V | TTT TAG TCG TGG GAC CTT G |
| 43 | Q229H | GCA AAA TTC ATA TCA AAG AAG |
| 44 | W327G | GCG GGA CCT GGG TAT CAT G |

TABLE 13-3

Table 13-3: PRIMER LIST
(No. IN THE LIST INDICATES SEQUENCE NUMBER)

| | |
|---|---|
| Name | Sequence |

| | | |
|---|---|---|
| 45 | F207V F | CAGGATGCATTTACAGTCATGTCAACCTTTGGTG |
| 46 | F207V R | CACCAAAGGTTGACATGACTGTAAATGCATCCTG |
| 47 | 2458 K83A F | GAACGAATACAAAGCAAGTTTGGGAAAC |
| 48 | 2458 K83A R | GTTTCCCAAACTTGCTTTGTATTCGTTC |
| 49 | 2458 Q229H F | GGGCAAAATTCATATCAAAGAAGCCG |
| 50 | 2458 Q229H R | CGGCTTCTTTGATATGAATTTTGCCC |
| 51 | 2458 V2571 F | CTTTGGTGACTCCATACAATTTTGG |
| 52 | 2458 V2571 R | CCAAAATTGTATGGAGTCACCAAAG |
| 53 | 2458 A301V F | GACGCGGAAGATGTTTATGGAACATTT |
| 54 | 2458 A301V R | AAATGTTCCATAAACATCTTCCGCGTC |
| 55 | 2458 D313E F | CCAATCGATTGAGGAAAAAAGCAAAAAAAAC |
| 56 | 2458 D313E R | GTTTTTTTTGCTTTTTTCCTCAATCGATTGG |
| 57 | 2458 A324V F | CTCGATTTTAGTCGTGGGACCTTGGTATC |
| 58 | 2458 A324V R | GATACCAAGGTCCCACGACTAAAATCGAG |
| 59 | 2458 L439V F | GCATCAAGGTGGGGTAATTCAAAACCG |
| 60 | 2458 L439V R | CGGTTTTGAATTACCCCACCTTGATGC |
| 61 | 2458 Q441E F | GGTGGGTTAATTGAAAACCGAACAC |
| 62 | 2458 Q441E R | GTGTTCGGTTTTCAATTAACCCACC |
| 63 | 2458 A537G F | GGTTTCGATAAAGGGCAGGCCTTGAC |
| 64 | 2458 A537G R | GTCAAGGCCTGCCCTTTATCGAAACC |
| 65 | 2458 N607K F | CACGATGTGAAGAATGCCACATACATCG |
| 66 | 2458 N607K R | CGATGTATGTGGCATTCTTCACATCGTG |
| 67 | T72A F | GAACGCCCTACGCGGTTTCTCC |
| 68 | T72A R | GGAGAAACCGCGTAGGGCGTTC |
| 69 | A137S F | CGGATACCTATGATTCGCTTGAATGGTTAC |
| 70 | A137S R | GTAACCATTCAAGCGAATCATAGGTATCCG |
| 71 | E551K S | AAG GTG AAT TTT AAA ATG CCA GAC |
| | | GTT GCG |
| 72 | E551K AS | CGC AAC GTC TGG CAT TTT AAA ATT |
| | | CAC CTT |
| 73 | M208A S | catttacattcgcgtcaacctttggtgtcc |
| 74 | M208A AS | ggacaccaaaggttgacgcgaatgtaaatg |
| 75 | 2458 G226S F | CGGATCAATTTAAGAGCAAAATTCAG |
| 76 | 2458 G226S R | CTGAATTTTGCTCTTAAATTGATCCG |
| 77 | F207H S | aggatgcatttacacacatgtcaacctttg |
| 78 | F207H AS | caaaggttgacatgtgtgtaaatgcatcct |

TABLE 13-4

Table 13-4: PRIMER LIST
(No. in the list indicates sequence number)

| | | | |
|---|---|---|---|
| No. | Name | MUTATION | Sequence |

| | | | |
|---|---|---|---|
| 79 | 2458 V184A F | V184A | CACAGGCTCCCGCAACAGACTGGTA |
| | | | TATC |
| 80 | 2458 V184A R | | GATATACCAGTCTGTTGCGGGAGCC |
| | | | TGTG |
| 81 | 2458 V184C F | V184C | CACAGGCTCCCTGCACAGACTGGTA |
| | | | TATC |
| 82 | 2458 V184C R | | GATATACCAGTCTGTGCAGGGAGCC |
| | | | TGTG |
| 83 | 2458 V184G F | V184G | CACAGGCTCCCGGCACAGACTGGTA |
| | | | TATC |
| 84 | 2458 V184G R | | GATATACCAGTCTGTGCCGGGAGCC |
| | | | TGTG |
| 85 | 2458 V184I F | V184I | CACAGGCTCCCATTACAGACTGGTA |
| | | | TATC |
| 86 | 2458 V184I R | | GATATACCAGTCTGTAATGGGAGCC |
| | | | TGTG |
| 87 | 2458 V184L F | V184L | CACAGGCTCCCCTAACAGACTGGTA |
| | | | TATC |
| 88 | 2458 V184L R | | GATATACCAGTCTGTTAGGGGAGCC |
| | | | TGTG |
| 89 | 2458 V184M F | V184M | CACAGGCTCCCATGACAGACTGGTA |
| | | | TATC |
| 90 | 2458 V184M R | | GATATACCAGTCTGTCATGGGAGCC |
| | | | TGTG |
| 91 | 2458 V184N F | V184N | CACAGGCTCCCAACACAGACTGGTA |
| | | | TATC |
| 92 | 2458 V184N R | | GATATACCAGTCTGTGTTGGGAGCC |
| | | | TGTG |
| 93 | 2458 V184P F | V184P | CACAGGCTCCCCAACAGACTGGTAT |
| | | | ATC |
| 94 | 2458 V184P R | | GATATACCAGTCTGTTGGGGGAGCC |
| | | | TGTG |
| 95 | 2458 V184S F | V184S | CACAGGCTCCCTCAACAGACTGGTA |
| | | | TATC |
| 96 | 2458 V184S R | | GATATACCAGTCTGTTGAGGGAGCC |
| | | | TGTG |
| 97 | 2458 V184T F | V184T | CACAGGCTCCCACAACAGACTGGTA |
| | | | TATC |
| 98 | 2458 V184T R | | GATATACCAGTCTGTTGTGGGAGCC |
| | | | TGTG |

TABLE 13-5

Table 13-5: PRIMER LIST
(No. IN THE LIST INDICATES SEQUENCE NUMBER)

| | | |
|---|---|---|
| No. | Name | Sequence |

| | | |
|---|---|---|
| 99 | 2458 | GAACGAATACAAAGCAAGTTTGGGAAAC |
| | K83A F | |
| 100 | 2458 | GGGCAAAATTCATATCAAAGAAGCCG |
| | Q229H F | |
| 101 | 2458 | CTTTGGTGACTCCATACAATTTTGG |
| | V2571 F | |
| 102 | 2458 | GACGCGGAAGATGTTTATGGAACATTT |
| | A3O1V F | |
| 103 | 2458 | CCAATCGATTGAGGAAAAAAGCAAAAAAAAC |
| | D313E F | |
| 104 | 2458 | CTCGATTTTAGTCGTGGGACCTTGGTATC |
| | A324V F | |
| 105 | 2458 | GCATCAAGGTGGGGTAATTCAAAACCG |
| | L439V F | |
| 106 | 2458 | GGTGGGTTAATTGAAAACCGAACAC |
| | Q441E F | |
| 107 | 2458 | GGTTTCGATAAAGGGCAGGCCTTGAC |
| | A537G F | |
| 108 | 2458 | CACGATGTGAAGAATGCCACATACATCG |
| | N607K F | |
| 109 | T72A F | GAACGCCCTACGCGGTTTCTCC |
| 110 | A137S F | CGGATACCTATGATTCGCTTGAATGGTTAC |
| 111 | Q229X F | GGGCAAAATTNNNATCAAAGAAGCCG |
| 112 | 1228X F+ | CAATTTAAGGGCAAANNNCCTATCAAAGAAGCCG |
| | Q229P F | |
| 113 | 1230X F+ | GGGCAAAATTCCTNNNAAAGAAGCCG |
| | Q229P F | |
| 114 | 1228X F+ | CAATTTAAGGGCAAANNNCATATCAAAGAAGCCG |
| | Q229H F | |
| 115 | 5256X F+ | CTTTGGTGACNNNATACAATTTTGGAATG |
| | V2571 F | |
| 116 | A137X F | CGGATACCTATGATNNNCTTGAATGGTTAC |
| 117 | 2458 | GGGCAAAATTCCTATCAAAGAAGCCG |
| | Q229P F | |
| 118 | A324X F | CAACTCGATTTTAGTCNNNGGACCTTGGTATC |
| 119 | A3O1X F | CTTTGACGCGGAAGATNNNTATGGAACATTTAAG |
| 120 | A537X F | GAAATGGTTTCGATAAANNNCAGGCCTTGACTCC |

TABLE 13-6

Table 13-6: PRIMER LIST
(No. IN THE LIST INDICATES SEQUENCE NUMBER)

| | | |
|---|---|---|
| No. | Name | Sequence |

| | | |
|---|---|---|
| 121 | Esp-S1 | CCGTAAGGAGGAATGTAGATGAAAAATACAATTTCGT |
| | | GCC |
| 122 | 5-AS1 | GGC TGC AGT TTG CGG GAT GGA AGG CCG GC |
| 123 | E-S1 | CCT CTA GAA TTT TTT CAA TGT GAT TT |
| 124 | Esp-AS1 | GCAGGAAATTGTATTTTTCATCTACATTCCTCCTTACG |
| | | GTGTTAT |
| 125 | EM1 | CTT ACA GAT GAC TAT AAT GTG ACT AAA AAC |
| 126 | EMR1 | GTT TTT AGT CAC ATT ATA GTC ATC TGT AAG |

### INDUSTRIAL APPLICABILITY

The present invention is useful in the fields concerning the methods for producing the peptides.

What is claimed is:

1. A mutant protein having an amino acid sequence comprising one or two or more mutations selected from any of the following mutations 1 to 38:
mutation 1: F207V,
mutation 2: Q441E,
mutation 3: K83A,
mutation 4: A301V,
mutation 5: V257I,
mutation 6: A537G,
mutation 7: A324V,
mutation 8: N607K,
mutation 9: D313E,
mutation 10: Q229H,
mutation 11: M208A,
mutation 12: E551K,
mutation 13: F207H,
mutation 14: T72A,
mutation 15: A137S,
mutation 16: L439V,
mutation 17: G226S,
mutation 18: D619E,
mutation 19: Y339H,
mutation 20: W327G,
mutation 21: V184A,
mutation 22: V184C,
mutation 23: V184G,
mutation 24: V184I,
mutation 25: V184L,
mutation 26: V184M,
mutation 27: V184P,
mutation 28: V184S,
mutation 29: V184T,
mutation 30: Q441K,
mutation 31: N442K,
mutation 32: D203N,
mutation 33: D203S,
mutation 34: F207A,
mutation 35: F207S,
mutation 36: Q441N,
mutation 37: F207T, and
mutation 38: F207I in an amino acid sequence of SEQ ID NO:2.

2. The mutant protein according to claim 1 further having one or several amino acid mutations selected from the group consisting of substitution, deletion, insertion, addition and inversion at positions other than one or two or more mutation positions of said mutations 1 to 38, and having a peptide-synthesizing activity.

3. The mutant protein according to claim 1 comprising at least the mutation 2.

4. The mutant protein according to claim 1 comprising at least the mutation 14.

5. A polynucleotide encoding an amino acid sequence of the mutant protein according to claim 1.

6. A recombinant polynucleotide comprising the polynucleotide according to claim 5.

7. A transformed microorganism comprising the recombinant polynucleotide according to claim 6.

8. A method for producing a mutant protein wherein the transformed microorganism according to claim 7 is cultured in a medium and said mutant protein is accumulated in the medium or the transformed microorganism.

9. A method for producing a peptide wherein a peptide synthesizing reaction is performed in the presence of the mutant protein according to claim 1.


SEQUENCE LISTING (126 sequences)

SEQ ID NO: 1 - DNA, 1935 bp, Sphingobacterium sp.
  CDS (61)..(1917): gene coding protein having peptide-forming activity
gaaaccaagtgtaaaattataatttacaccaaagaatgtactgaacaaataattatctgaatgaaaaatacaatttcgtgcctaactttagcgcttttaagcgcaagccagttacatgctcaaacagctgccgactcggcttatgttagagatcattatgaaaagaccgaagtagcaattcccatgcgagatgggaaaaaattatttactgcgatctacagtccaaaagacaaatccaagaaatatccagttttgctcaatagaacgccctacacggtttcaccttatgggcagaacgaatataaaaaaagcttgggaaactttccccaaatgatgcgtgaaggctatattttcgtttaccaggatgtccgtggcaagtggatgagcgaaggtgattttgaagatatacgtccgaccacgtacagcaaagataaaaaagcaatcgatgaaagtacggatacctatgatgcgcttgaatggttacagaaaaatctcaaaaactataatggcaaagccgggctctatgggatttcctatccaggcttctattctaccgtcggattggtcaaaacacacccgagcttgaaggcagtctccccacaggctcccgtaacagactggtatatcggcgacgacttccaccataatggcgtattgtttcttcaggatgcatttacattcatgtcaacctttggtgtccctcgtccaaaacccattacaccggatcaatttaagggcaaaattcagatcaaagaagccgataaatataacttttttgcagaagcaggaacagcgcgggaactcaaagaaaagtattttggtgactccgtacaattttggaatgacctgtttaagcatcccgactatgatgatttttggaaatcgcgtgtgatcacgaattctttacaggaggtaaaaccagctgtgatggtggttggtggtttctttgacgcggaagatgcttatggaacatttaagacctaccaatcgattgaggataaaagcaaaaaaaacaactcgattttagtcgcgggaccttggtatcatggcggttgggttcgtgcagaaggaaactatttaggtgatatccaatttgagaaaaaaaccagtattacttatcaggaacaatttgaacaaccatttttcaaatattacctaaaagatgaaggaaacttcgccccttccgaagctaacatttttgtttcaggcagcaacgaatggaaacatttcgaacagtggccaccaaaaaatgtagagacaaaaaaactatacttccaacctcaggggaaacttggatttgacaaagttcaacgtacagattcctgggatgaatatgtaacagaccctaataaacctgttccgcatcaaggtgggttaattcaaaaccgaacacgggagtatatggtagatgatcaacgtttcgcggctagtcgccctgatgtcatggtttatcaaacggaaccgttgacggaggacctgacgatagtaggcccaatcaaaaactttctcaaagtttcttcaacaggaacagacgcggactatgttgtcaaactgattgacgtttatccgaatgatgcagcaagttatcaaggaaaaacaatggctggatatcaaatgatggtacgtggtgagatcatggcggggaaataccgaaatggtttcgataaagcgcaggccttgactccaggtatggtcgaaaaggtgaattttgaaatgccagacgttgcgcataccttcaaaaaaggacatcgcattatggttcaggtacaaaactcatggtttccgctggcagaacgaaatccacaggtgtttttagcaccttatacagctaccaaagctgatttccgcaaagctacccaacgtatttttcacgatgtgaacaatgccacatacatcgaattttctgtcctcaaagattagcaggtaaattcgaaa

SEQ ID NO: 2 - protein, 619 aa, Sphingobacterium sp.
MKNTISCLTLALLSASQLHAQTAADSAYVRDHYEKTEVAIPMRDGKKLFTAIYSPKDKSKKYPVLLNRTPYTVSPYGQNEYKKSLGNFPQMMREGYIFVYQDVRGKWMSEGDFEDIRPTTYSKDKKAIDESTDTYDALEWLQKNLKNYNGKAGLYGISYPGFYSTVGLVKTHPSLKAVSPQAPVTDWYIGDDFHHNGVLFLQDAFTFMSTFGVPRPKPITPDQFKGKIQIKEADKYNFFAEAGTARELKEKYFGDSVQFWNDLFKHPDYDDFWKSRVITNSLQEVKPAVMVVGGFFDAEDAYGTFKTYQSIEDKSKKNNSILVAGPWYHGGWVRAEGNYLGDIQFEKKTSITYQEQFEQPFFKYYLKDEGNFAPSEANIFVSGSNEWKHFEQWPPKNVETKKLYFQPQGKLGFDKVQRTDSWDEYVTDPNKPVPHQGGLIQNRTREYMVDDQRFAASRPDVMVYQTEPLTEDLTIVGPIKNFLKVSSTGTDADYVVKLIDVYPNDAASYQGKTMAGYQMMVRGEIMAGKYRNGFDKAQALTPGMVEKVNFEMPDVAHTFKKGHRIMVQVQNSWFPLAERNPQVFLAPYTATKADFRKATQRIFHDVNNATYIEFSVLKD

SEQ ID NO: 3 - DNA, 30 bp, Artificial Sequence Sequence
  Synthetic DNA
cgcgaattcatgaaaaatacaatttcgtgc

SEQ ID NO: 4 - DNA, 33 bp, Artificial Sequence Sequence
  primer
cgcctgcagctaatctttgaggacagaaaattc

SEQ ID NO: 5 - DNA, 30 bp, Artificial Sequence
  primer
gggaattccatatgaaaaatacaatttcgt

SEQ ID NO: 6 - DNA, 29 bp, Artificial Sequence
  primer
gctctagactaatctttgaggacagaaaa

SEQ ID NO: 7 - DNA, 19 bp, Artificial Sequence
  primer
tgctcaatagaacgcccta

SEQ ID NO: 8 - DNA, 19 bp, Artificial Sequence
  primer
ccgagcttgaaggcagtct

SEQ ID NO: 9 - DNA, 19 bp, Artificial Sequence
  primer
acgcggaagatgcttatgg

SEQ ID NO: 10 - DNA, 18 bp, Artificial Sequence
  primer
aagttcaacgtacagatt

SEQ ID NO: 11 - DNA, 20 bp, Artificial Sequence
  primer
ggtatccgtactttcatcga

SEQ ID NO: 12 - DNA, 35 bp, Artificial Sequence
  primer
gcatttacattcatggacacctttggtgtccctcg

SEQ ID NO: 13 - DNA, 33 bp, Artificial Sequence
  primer
caaggtgggttaattgaaaaccgaacacgggag

SEQ ID NO: 14 - DNA, 33 bp, Artificial Sequence
  primer
caaggtgggttaattaaaaaccgaacacgggag

SEQ ID NO: 15 - DNA, 36 bp, Artificial Sequence
  primer
ggtgggttaattcaaaaacgaacacgggagtatatg

SEQ ID NO: 16 - DNA, 31 bp, Artificial Sequence
  primer
caaaaccgaacagaggagtatatggtagatg

SEQ ID NO: 17 - DNA, 31 bp, Artificial Sequence
  primer
caaaaccgaacatttgagtatatggtagatg

SEQ ID NO: 18 - DNA, 33 bp, Artificial Sequence
  primer
gtattgtttcttcagaatgcatttacattcatg

SEQ ID NO: 19 - DNA, 33 bp, Artificial Sequence
  primer
gtattgtttcttcagtctgcatttacattcatg

SEQ ID NO: 20 - DNA, 34 bp, Artificial Sequence
  primer
caggatgcatttacagccatgtcaacctttggtg

SEQ ID NO: 21 - DNA, 34 bp, Artificial Sequence
  primer
caggatgcatttacatccatgtcaacctttggtg

SEQ ID NO: 22 - DNA, 34 bp, Artificial Sequence
  primer
gcatttacattcatggcaacctttggtgtccctc

SEQ ID NO: 23 - DNA, 33 bp, Artificial Sequence
  primer
caaggtgggttaattaacaaccgaacacgggag

SEQ ID NO: 24 - DNA, 33 bp, Artificial Sequence
  primer
caaggtgggttaattgacaaccgaacacgggag

SEQ ID NO: 25 - DNA, 30 bp, Artificial Sequence
  primer
cagaacgaatacaaagcaagtttgggaaac

SEQ ID NO: 26 - DNA, 34 bp, Artificial Sequence
  primer
caggatgcatttacagtcatgtcaacctttggtg

SEQ ID NO: 27 - DNA, 34 bp, Artificial Sequence
  primer
caggatgcatttacaggcatgtcaacctttggtg

SEQ ID NO: 28 - DNA, 34 bp, Artificial Sequence
  primer
caggatgcatttacaaccatgtcaacctttggtg

SEQ ID NO: 29 - DNA, 33 bp, Artificial Sequence
  primer
gatgcatttacattcgcgtcaacctttggtgtc

SEQ ID NO: 30 - DNA, 32 bp, Artificial Sequence
  primer
gcatttacattcatgggaacctttggtgtccc

SEQ ID NO: 31 - DNA, 34 bp, Artificial Sequence
  primer
caggatgcatttacaatcatgtcaacctttggtg

SEQ ID NO: 32 - DNA, 33 bp, Artificial Sequence
  primer
gattttgaagatatagctccgaccacgtacagc

SEQ ID NO: 33 - DNA, 34 bp, Artificial Sequence
  primer
caggatgcatttacagtcatggcaacctttggtg

SEQ ID NO: 34 - DNA, 21 bp, Artificial Sequence
  primer
caaggtggggtaattcaaaac

SEQ ID NO: 35 - DNA, 19 bp, Artificial Sequence
  primer
cgataaagggcaggccttg

SEQ ID NO: 36 - DNA, 20 bp, Artificial Sequence
  primer
gcggaagatgtttatggaac

SEQ ID NO: 37 - DNA, 19 bp, Artificial Sequence
  primer
caatttaagagcaaaattc

SEQ ID NO: 38 - DNA, 20 bp, Artificial Sequence
  primer
ggtgactccatacaattttg

SEQ ID NO: 39 - DNA, 20 bp, Artificial Sequence
  primer
tttctgtcctcaaagaatag

SEQ ID NO: 40 - DNA, 19 bp, Artificial Sequence
  primer
gaaggaaaccatttaggtg

SEQ ID NO: 41 - DNA, 20 bp, Artificial Sequence
  primer
cacgatgtgaagaatgccac

SEQ ID NO: 42 - DNA, 19 bp, Artificial Sequence
  primer
ttttagtcgtgggaccttg

SEQ ID NO: 43 - DNA, 21 bp, Artificial Sequence
  primer
gcaaaattcatatcaaagaag

SEQ ID NO: 44 - DNA, 19 bp, Artificial Sequence
  primer
gcgggacctgggtatcatg

SEQ ID NO: 45 - DNA, 34 bp, Artificial Sequence; identical to SEQ ID NO: 26
  primer

SEQ ID NO: 46 - DNA, 34 bp, Artificial Sequence
  primer
caccaaaggttgacatgactgtaaatgcatcctg

SEQ ID NO: 47 - DNA, 28 bp, Artificial Sequence
  primer
gaacgaatacaaagcaagtttgggaaac

SEQ ID NO: 48 - DNA, 28 bp, Artificial Sequence
  primer
gtttcccaaacttgctttgtattcgttc

SEQ ID NO: 49 - DNA, 26 bp, Artificial Sequence
  primer
gggcaaaattcatatcaaagaagccg

SEQ ID NO: 50 - DNA, 26 bp, Artificial Sequence
  primer
cggcttctttgatatgaattttgccc

SEQ ID NO: 51 - DNA, 25 bp, Artificial Sequence
  primer
ctttggtgactccatacaattttgg

SEQ ID NO: 52 - DNA, 25 bp, Artificial Sequence
  primer
ccaaaattgtatggagtcaccaaag

SEQ ID NO: 53 - DNA, 27 bp, Artificial Sequence
  primer
gacgcggaagatgtttatggaacattt

SEQ ID NO: 54 - DNA, 27 bp, Artificial Sequence
  primer
aaatgttccataaacatcttccgcgtc

SEQ ID NO: 55 - DNA, 31 bp, Artificial Sequence
  primer
ccaatcgattgaggaaaaaagcaaaaaaaac

SEQ ID NO: 56 - DNA, 31 bp, Artificial Sequence
  primer
gttttttttgcttttttcctcaatcgattgg

SEQ ID NO: 57 - DNA, 29 bp, Artificial Sequence
  primer
ctcgattttagtcgtgggaccttggtatc

SEQ ID NO: 58 - DNA, 29 bp, Artificial Sequence
  primer
gataccaaggtcccacgactaaaatcgag

SEQ ID NO: 59 - DNA, 27 bp, Artificial Sequence
  primer
gcatcaaggtggggtaattcaaaaccg

SEQ ID NO: 60 - DNA, 27 bp, Artificial Sequence
  primer
cggttttgaattaccccaccttgatgc

SEQ ID NO: 61 - DNA, 25 bp, Artificial Sequence
  primer
ggtgggttaattgaaaaccgaacac

SEQ ID NO: 62 - DNA, 25 bp, Artificial Sequence
  primer
gtgttcggttttcaattaacccacc

SEQ ID NO: 63 - DNA, 26 bp, Artificial Sequence
  primer
ggtttcgataaagggcaggccttgac

SEQ ID NO: 64 - DNA, 26 bp, Artificial Sequence
  primer
gtcaaggcctgccctttatcgaaacc

SEQ ID NO: 65 - DNA, 28 bp, Artificial Sequence
  primer
cacgatgtgaagaatgccacatacatcg

SEQ ID NO: 66 - DNA, 28 bp, Artificial Sequence
  primer
cgatgtatgtggcattcttcacatcgtg

SEQ ID NO: 67 - DNA, 22 bp, Artificial Sequence
  primer
gaacgccctacgcggtttctcc

SEQ ID NO: 68 - DNA, 22 bp, Artificial Sequence
  primer
ggagaaaccgcgtagggcgttc

SEQ ID NO: 69 - DNA, 30 bp, Artificial Sequence
  primer
cggatacctatgattcgcttgaatggttac

SEQ ID NO: 70 - DNA, 30 bp, Artificial Sequence
  primer
gtaaccattcaagcgaatcataggtatccg

SEQ ID NO: 71 - DNA, 30 bp, Artificial Sequence
  primer
aaggtgaattttaaaatgccagacgttgcg

SEQ ID NO: 72 - DNA, 30 bp, Artificial Sequence
  primer
cgcaacgtctggcattttaaaattcacctt

SEQ ID NO: 73 - DNA, 30 bp, Artificial Sequence
  primer
catttacattcgcgtcaacctttggtgtcc

SEQ ID NO: 74 - DNA, 30 bp, Artificial Sequence
  primer
ggacaccaaaggttgacgcgaatgtaaatg

SEQ ID NO: 75 - DNA, 26 bp, Artificial Sequence
  primer
cggatcaatttaagagcaaaattcag

SEQ ID NO: 76 - DNA, 26 bp, Artificial Sequence
  primer
ctgaattttgctcttaaattgatccg

SEQ ID NO: 77 - DNA, 30 bp, Artificial Sequence
  primer
aggatgcatttacacacatgtcaacctttg

SEQ ID NO: 78 - DNA, 30 bp, Artificial Sequence
  primer
caaaggttgacatgtgtgtaaatgcatcct

SEQ ID NO: 79 - DNA, 29 bp, Artificial Sequence
  primer
cacaggctcccgcaacagactggtatatc

SEQ ID NO: 80 - DNA, 29 bp, Artificial Sequence
  primer
gatataccagtctgttgcgggagcctgtg

SEQ ID NO: 81 - DNA, 29 bp, Artificial Sequence
  primer
cacaggctccctgcacagactggtatatc

SEQ ID NO: 82 - DNA, 29 bp, Artificial Sequence
  primer
gatataccagtctgtgcagggagcctgtg

SEQ ID NO: 83 - DNA, 29 bp, Artificial Sequence
  primer
cacaggctcccggcacagactggtatatc

SEQ ID NO: 84 - DNA, 29 bp, Artificial Sequence
  primer
gatataccagtctgtgccgggagcctgtg

SEQ ID NO: 85 - DNA, 29 bp, Artificial Sequence
  primer
cacaggctcccattacagactggtatatc

SEQ ID NO: 86 - DNA, 29 bp, Artificial Sequence
  primer
gatataccagtctgtaatgggagcctgtg

SEQ ID NO: 87 - DNA, 29 bp, Artificial Sequence
  primer
cacaggctcccctaacagactggtatatc

SEQ ID NO: 88 - DNA, 29 bp, Artificial Sequence
  primer
gatataccagtctgttaggggagcctgtg

SEQ ID NO: 89 - DNA, 29 bp, Artificial Sequence
  primer
cacaggctcccatgacagactggtatatc

SEQ ID NO: 90 - DNA, 29 bp, Artificial Sequence
  primer
gatataccagtctgtcatgggagcctgtg

SEQ ID NO: 91 - DNA, 29 bp, Artificial Sequence
  primer
cacaggctcccaacacagactggtatatc

SEQ ID NO: 92 - DNA, 29 bp, Artificial Sequence
  primer
gatataccagtctgtgttgggagcctgtg

SEQ ID NO: 93 - DNA, 28 bp, Artificial Sequence
  primer
cacaggctccccaacagactggtatatc

SEQ ID NO: 94 - DNA, 29 bp, Artificial Sequence
  primer
gatataccagtctgttgggggagcctgtg

SEQ ID NO: 95 - DNA, 29 bp, Artificial Sequence
  primer
cacaggctccctcaacagactggtatatc

SEQ ID NO: 96 - DNA, 29 bp, Artificial Sequence
  primer
gatataccagtctgttgagggagcctgtg

SEQ ID NO: 97 - DNA, 29 bp, Artificial Sequence
  primer
cacaggctcccacaacagactggtatatc

SEQ ID NO: 98 - DNA, 29 bp, Artificial Sequence
  primer
gatataccagtctgttgtgggagcctgtg

SEQ ID NO: 99 - DNA, 28 bp, Artificial Sequence
  primer
gaacgaatacaaagcaagtttgggaaac

SEQ ID NO: 100 - DNA, 26 bp, Artificial Sequence
  primer
gggcaaaattcatatcaaagaagccg

SEQ ID NO: 101 - DNA, 25 bp, Artificial Sequence
  primer
ctttggtgactccatacaattttgg

SEQ ID NO: 102 - DNA, 27 bp, Artificial Sequence
  primer
gacgcggaagatgtttatggaacattt

SEQ ID NO: 103 - DNA, 31 bp, Artificial Sequence; identical to SEQ ID NO: 55
  primer

SEQ ID NO: 104 - DNA, 29 bp, Artificial Sequence
  primer
ctcgattttagtcgtgggaccttggtatc

SEQ ID NO: 105 - DNA, 27 bp, Artificial Sequence
  primer
gcatcaaggtggggtaattcaaaaccg

SEQ ID NO: 106 - DNA, 25 bp, Artificial Sequence
  primer
ggtgggttaattgaaaaccgaacac

SEQ ID NO: 107 - DNA, 26 bp, Artificial Sequence
  primer
ggtttcgataaagggcaggccttgac

SEQ ID NO: 108 - DNA, 28 bp, Artificial Sequence
  primer
cacgatgtgaagaatgccacatacatcg

SEQ ID NO: 109 - DNA, 22 bp, Artificial Sequence
  primer
gaacgccctacgcggtttctcc

SEQ ID NO: 110 - DNA, 30 bp, Artificial Sequence; identical to SEQ ID NO: 69
  primer

SEQ ID NO: 111 - DNA, 26 bp, Artificial Sequence
  primer
gggcaaaattnnnatcaaagaagccg

SEQ ID NO: 112 - DNA, 34 bp, Artificial Sequence
  primer
caatttaagggcaaannncctatcaaagaagccg

SEQ ID NO: 113 - DNA, 26 bp, Artificial Sequence
  primer
gggcaaaattcctnnnaaagaagccg

SEQ ID NO: 114 - DNA, 34 bp, Artificial Sequence
  primer
caatttaagggcaaannncatatcaaagaagccg

SEQ ID NO: 115 - DNA, 29 bp, Artificial Sequence
  primer
ctttggtgacnnnatacaattttggaatg

SEQ ID NO: 116 - DNA, 30 bp, Artificial Sequence
  primer
cggatacctatgatnnncttgaatggttac

SEQ ID NO: 117 - DNA, 26 bp, Artificial Sequence
  primer
gggcaaaattcctatcaaagaagccg

SEQ ID NO: 118 - DNA, 32 bp, Artificial Sequence
  primer
caactcgattttagtcnnnggaccttggtatc

SEQ ID NO: 119 - DNA, 34 bp, Artificial Sequence
  primer
ctttgacgcggaagatnnntatggaacatttaag

SEQ ID NO: 120 - DNA, 34 bp, Artificial Sequence
  primer
gaaatggtttcgataaannncaggccttgactcc

SEQ ID NO: 121 - DNA, 40 bp, Artificial Sequence
  primer
ccgtaaggaggaatgtagatgaaaaatacaatttcgtgcc

SEQ ID NO: 122 - DNA, 29 bp, Artificial Sequence
  primer
ggctgcagtttgcgggatggaaggccggc

SEQ ID NO: 123 - DNA, 26 bp, Artificial Sequence
  primer
cctctagaattttttcaatgtgattt

SEQ ID NO: 124 - DNA, 45 bp, Artificial Sequence
  primer
gcaggaaattgtatttttcatctacattcctccttacggtgttat

SEQ ID NO: 125 - DNA, 30 bp, Artificial Sequence
  primer
cttacagatgactataatgtgactaaaaac

SEQ ID NO: 126 - DNA, 30 bp, Artificial Sequence
  primer
gtttttagtcacattatagtcatctgtaag